(19) United States
(12) Patent Application Publication
(10) Pub. No.: US 2015/0132845 A1
(43) Pub. Date: May 14, 2015

(54) DEVICE AND METHOD FOR CONTINUOUS CELL CULTURE AND OTHER REACTIONS

(71) Applicant: Massachusetts Institute of Technology, Cambridge, MA (US)

(72) Inventors: Rajeev Jagga Ram, Arlington, MA (US); Kevin Shao-Kwan Lee, Cambridge, MA (US)

(73) Assignee: Massachusetts Institute of Technology, Cambridge, MA (US)

(21) Appl. No.: 14/535,048

(22) Filed: Nov. 6, 2014

Related U.S. Application Data
(63) Continuation of application No. 13/249,959, filed on Sep. 30, 2011, now abandoned.

(51) Int. Cl. C12M 1/00; C12M 1/34; C12M 1/04

(52) U.S. Cl. CPC C12M 23/26; C12M 23/24; C12M 41/40; C12M 29/18
USPC 435/348; 435/293.1; 435/252.8; 435/420; 435/383; 435/349; 435/363; 435/350; 435/351; 435/353; 435/354; 435/366; 435/358; 435/255.1

(57) ABSTRACT

Devices, systems, and methods for continuous cell culture and other reactions are generally described. In some embodiments, chambers (e.g., cell growth chambers) including at least a portion of a wall formed of a flexible member are provided. A retaining structure can be incorporated outside and proximate to the chamber such that when liquid is added to the chamber, the flexible member is consistently and predictably deformed, and a consistent volume of liquid is added. The flexible member can be formed of, in some embodiments, a gas-permeable medium. In some embodiments, reaction chambers can be arranged in a fluidic loop, and a bypass channel can be used to introduce and/or extract fluid from the loop without affecting loop operation.

### RELATED APPLICATIONS

This application is a continuation of U.S. patent application Ser. No. 13/249,959, filed Sep. 30, 2011, and entitled “Device and Method for Continuous Cell Culture and Other Reactions,” which is incorporated herein by reference in its entirety for all purposes.

### GOVERNMENT SPONSORSHIP

This invention was made with government support under Grant No. DBI0649879, awarded by the National Science Foundation. The government has certain rights in the invention.

### TECHNICAL FIELD

Devices, systems, and methods for continuous cell culture and other reactions are generally described.

### BACKGROUND

Understanding cell behavior is important in microbial physiology, genetics, ecology, and biotechnology. Growth kinetics, or the relationship between cell growth rate and nutrient supply, plays an important role in the understanding of cell function. While research has been focused on understanding growth kinetics from a genomic level, there is still great difficulty in making the leap from genetic analysis to accurate verification with controlled cell growth experiments, or cell cultures. Most culture systems operate as batch cultures, providing a fixed amount of nutrients and oxygenation for the initial cells and supporting cell growth until it becomes limited by either a nutrient source or oxygen. Batch cultures are generally not ideal for characterizing cellular processes since cells are constantly subjected to environmental changes such as changes in acidity, oxygen content, or even increased cell population. It has been recognized that, in order to study bacterial growth with precision, a constant and controllable environment is necessary.

Continuous culture under steady state conditions can provide results that are much less sensitive to operator variation and can lead to more reproducible results. One example of a continuous culture system is a chemostat. Chemostats are bioreactors to which fresh medium is continuously added, while culture liquid is continuously removed to keep the culture volume constant. By changing the rate with which medium is added to the bioreactor, the growth rate of the microorganism within the reactor can be controlled. Unlike batch culture, where growth time scales are hours to days, continuous culture systems can be run for days or weeks at steady state. In some continuous growth systems, including chemostats, the bioreactor is operated such that there is constant volume of liquid within the bioreactor. However, current methods make it difficult to control liquid volume and growth conditions in these systems. Accordingly, improved systems and methods are desired.

### SUMMARY

Devices, systems, and methods for continuous cell culture and other reactions are provided. The subject matter of the present invention involves, in some cases, interrelated products, alternative solutions to a particular problem, and/or a plurality of different uses of one or more systems and/or articles.

In one set of embodiments, an article for transporting liquid is provided. In some embodiments, the article comprises a chamber with at least a portion of a wall of the chamber defined by a flexible member; a first inlet fluidically connected to the chamber and constructed and arranged to deliver a first liquid containing a reactant to the chamber; and an outlet fluidically connected to the chamber. In some embodiments, the flexible member is configured to substantially conform to the shape of a retaining structure outside the chamber and adjacent the flexible member when a pressure within the chamber is greater than a pressure outside the chamber.

A device for performing a chemical or biochemical reaction is provided, in some embodiments. The device comprises, in certain embodiments, a first fluidic pathway arranged in a continuous loop; a bypass channel connected to first and second portions of the first fluidic pathway; a first valve positioned between the bypass channel and the first portion of the first fluidic pathway; and a second value positioned between the bypass channel and the second portion of the first fluidic pathway. In some embodiments, when the first and second valves are closed, the first fluidic pathway is isolated from the bypass channel, and when the first and second valves are opened a second fluidic pathway is formed by the first and second portions of the first fluidic pathway and the bypass channel.

In some embodiments, a method of operating a chemical or biochemical reactor is provided. The method comprises, in certain embodiments, transporting a first liquid comprising a reactant into a chamber, wherein at least a portion of a wall of the chamber is defined by a gas-permeable medium, such that at least a portion of the first liquid exits the chamber via the gas-permeable medium; and transporting a second liquid into the chamber to increase the liquid volume in the chamber and decrease the concentration of the reactant within the chamber.

A method of performing a chemical or biochemical reaction is provided, in some embodiments. The method comprises, in certain embodiments, providing a first liquid comprising a reactant to a chamber via a first inlet, the chamber having at least a portion of a wall of the chamber defined by a flexible member; closing the first inlet; and providing a second liquid to the chamber via a second inlet such that, after the second liquid has been provided to the chamber, the flexible member has been deflected such that the shape of the flexible member substantially conforms to the shape of a retaining structure outside the chamber and adjacent the flexible member.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1C are exemplary cross-sectional schematic illustrations of chamber configurations, according to one set of embodiments;

FIGS. 2A-2B are, according to some embodiments, exemplary top-view schematic illustrations of reactor systems;

FIGS. 3A-3C are exemplary schematic diagrams and a photograph of a reactor system, according to one set of embodiments;

FIG. 4 is, according to one set of embodiments, a schematic diagram of a reactor system;

FIG. 5 is a plot of 99% mixing time as a function of mixing period, a schematic diagram of mixing sections, and associated photographs, according to one set of embodiments;

FIG. 6 includes plots of k_{L}a as a function of mixer period and pressure, as well as an associated schematic diagram of a portion of a reactor system, according to one set of embodiments;

FIG. 7 includes a plot of injection volume as a function of external fluid pressure, and an associated schematic diagram of a reactor system, according to one set of embodiments;

FIG. 8 includes plots of glucose input, optical density, pH, O₂ percentage, and flow rate as a function of time, according to some embodiments; and

FIGS. 9A-9D are plots of concentration, flow rate, optical density, and glucose input as a function of time, according to some embodiments.

### DETAILED DESCRIPTION

Devices, systems, and methods for continuous cell culture and other reactions are generally described. In some embodiments, chambers (e.g., cell growth chambers) including at least a portion of a wall formed of a flexible member are provided. In some embodiments, a retaining structure can be incorporated outside and proximate to the chamber such that when liquid is added to the chamber, the flexible member is consistently and predictably deformed, and a consistent volume of liquid is added. The flexible member can be formed of, in some embodiments, a gas-permeable medium. In some embodiments, reaction chambers can be arranged in a fluidic loop, and a bypass channel can be used to introduce and/or extract fluid from the loop without affecting loop operation.

In many reactor systems (including, for example, chemostats and other cell culture systems), it is desirable to maintain a constant liquid volume within the reactor. Many such systems include reactors in which at least a portion of a wall (or walls) is formed of a gas-permeable medium. For example, in many cell growth systems, the cell growth chamber includes at least a portion of a wall formed of a gas-permeable medium such that oxygen (for aerobic growth) or other gases that are needed to grow cells can be delivered. When reaction chamber walls are formed of a gas-permeable medium, some of the liquid (e.g., growth medium) within the reaction chamber can escape during operation, for example, via evaporation. Accordingly, constant volume can be difficult to maintain.

One way to address the loss of liquid within the reaction chamber is to periodically re-fill the reaction chamber with liquid to re-establish a constant volume. However, this process can be difficult when reaction chambers comprising gas-permeable walls are employed. Reaction chambers often employ relatively thin gas-permeable walls (which can be flexible) in order to enhance gas transport through the wall. When liquid is added to a chamber with thin, flexible walls, inconsistent deformation in the wall can lead to varying amounts of liquid added to the chamber (depending upon the pressure at which the liquid is added and the stress-strain behavior of the flexible wall material). Accordingly, maintaining constant liquid volumes within such reaction chambers can be difficult.

In some embodiments, a chamber with a wall defined at least in part by a flexible member can be configured such that the amount of liquid within the chamber can be easily reset to a substantially constant volume by flowing an inlet liquid into the chamber, regardless of the pressure at which the inlet liquid is provided. This can be achieved, for example, by incorporating a substantially rigid retaining structure outside the chamber and adjacent the flexible member to restrict the movement of the flexible member. In some such embodiments, when makeup liquid is provided at a higher pressure than the pressure above the flexible member, the flexible member expands only until it comes into contact with the retaining structure, after which further pressure results in minimal or no deformation of the flexible member with respect to chamber volume. In this way, the retaining structure defines a nominally maximum volume the chamber may occupy when an inlet liquid is supplied to the chamber, regardless of the pressure at which the inlet liquid is supplied.

In some embodiments, a chamber with a wall defined at least in part by a flexible member also includes a second wall, which can be substantially rigid. In some such embodiments, the second wall can be configured such that the flexible member is able to flex and conform to the second wall, reducing the volume of the chamber to zero or nearly zero, for example, when the pressure outside the chamber is larger than the sum of the pressure inside the chamber and the pressure required to conform the flexible member to the second wall. Such configurations can be desirable, for example, in situations where a chamber must be filled with minimal introduction of bubbles, in situations where a chamber must be filled but only contains a single inlet and no outlet to vent the gas in the chamber, in situations where the full chamber contents are desired for extraction, and/or in situations where multiple chambers are interconnected and the full volume of one chamber should be moved to the adjacent connected chamber. In some such embodiments, one is able to force the flexible member to conform to the second wall of the chamber, expelling all or nearly all of the contents of the chamber through a connected outlet channel.

Some previous reaction systems (e.g., cell culture systems) have employed reaction areas arranged in a fluidic loop. In some such systems, mixing is maintained during the reaction (e.g., cell growth) by transporting liquid around the loop. In many such systems, removal of the reaction products (e.g., cells) produced in the reaction loop requires that flow within the loop be discontinued while individual portions of the loop are evacuated and rinsed. Cutting off flow within the reaction loop generally prevents continuous operation of the reactor. Accordingly, in one set of embodiments, a bypass channel can fluidically connect two portions of a reaction loop. In some embodiments, liquid is circulated along a first path including the bypass channel and the reaction chamber loop during a first period of time, and liquid is circulated along a second path including the reaction chamber loop but not the bypass channel during a second period of time different from the first period of time.

In addition, continuous reaction systems usually include an inlet to supply new reactants and an outlet to remove sample and waste. In systems with a flexible member, difficulty can arise since the flexible member is able to deform and accommodate the additionally injected volume. In many situations (including some instances when multiple chambers with flexible members are arranged in a connected fluidic pathway, such as a fluidic loop, for mixing), it is desirable for the volume inside one and/or more chambers during operation to be less than the maximum volume available within the chamber(s) (e.g., when the flexible member is deflected to conform to the upper wall). In some embodiments, the problem of accumulating additional volume within a chamber, for example due to the input fluid increasing the chamber volume instead of pushing an equivalent volume of fluid through the outlet, a bypass channel can be employed. In some embodiments, the bypass channel is designed to have substantially rigid walls (e.g., including no flexible member) such that any fluid entering the bypass results in the same volume of fluid leaving the bypass. In some embodiments, the inlet(s) from the source fluid and/or the outlets from the reaction system are directly fluidically connected only to the bypass channel. In some embodiments, the bypass channel can be isolated from the chamber(s) forming the fluidic loop, for example, by positioning valves between the bypass channel and the chamber(s). In some such embodiments, the volume entering the bypass channel will be substantially equal to the volume exiting the bypass channel. After volume exchange has taken place in the bypass, the inlet and outlet to the bypass may be closed, and the bypass can be reconnected to the chamber(s), allowing mixing of the newly introduced bypass fluid with the fluid in the chamber(s).

While a continuous cell culture system is described generally throughout, the description of cell culture is meant to be exemplary, and the present invention is not so limited. In addition to continuous cell culture, the articles, systems, and methods described herein can be used in a variety of other chemical reactor systems including chemical synthesis reactors, photobioreactors, sewage treatment reactors, bioreactors from which cell products are harvested, evolution systems, cell isolators and/or selectors, and the like.

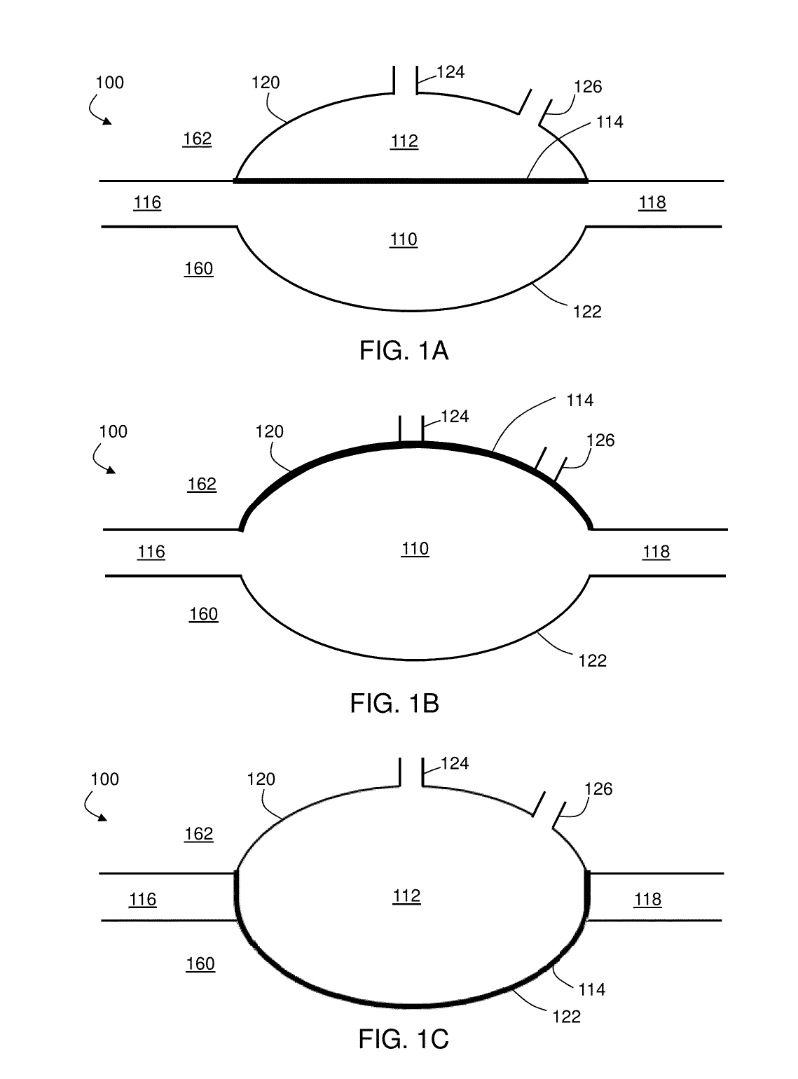

FIGS. 1A-1C include exemplary cross-sectional schematic illustrations of chamber configurations that can be used in association with the embodiments described herein. In FIG. 1A, portion 100 of a reaction system (e.g., a cell culture system) includes a first chamber 110 and a second chamber 112. At least a portion of a wall of the first chamber 110 can be defined by a flexible member 114. Flexible member can comprise, for example, a gas-permeable medium such as a gas-permeable polymer. The flexible member may be elastic in some embodiments. For example, the flexible member may be configured (e.g., to include suitable dimensions and/or materials) such that the flexible member does not undergo plastic deformation during operation of the device. In some embodiments, at least a portion of a wall of second chamber 112 is defined by flexible member 114. In FIGS. 1A-1C, portion 100 also includes a first inlet 116 fluidically connected to chamber 110. First inlet 116 can be configured to deliver a first liquid containing a reactant (e.g., cell culture medium or any other suitable reactant) to chamber 110. Portion 100 can also include outlet 118 fluidically connected to chamber 110. Outlet 118 can be configured to transport the contents of chamber 110 away from chamber 110, for example, during mixing and/or after the reaction within chamber 110 has proceeded to the desired extent and reaction product is to be harvested.

Portion 100 can also include retaining structure 120, which can be positioned outside chamber 110. Retaining structure 120 can be used to control the extent to which flexible member 114 is deformed when a pressure within chamber 110 is greater than a pressure outside chamber 110 (e.g., by closing outlet 118 or another channel downstream of chamber 110 and adding a liquid to chamber 110, by applying a vacuum to chamber 112, or by any other suitable method). In FIGS. 1A-1C, retaining structure 120 forms a wall of chamber 112. In other embodiments, however, retaining structure 120 is a standalone structure, and does not form all or part of a wall of chamber 112.

Flexible member 114 can be made from a gas-permeable medium. By fabricating flexible member 114 from a gas-permeable medium, a gas (e.g., oxygen, dry air, or any other suitable gas) can be added to chamber 110 during operation of the reaction system, for example by transporting the gas into chamber 112 through inlet 124 and subsequently across flexible member 114. This can be useful, for example, when growing cells or performing any other chemical or biochemical reaction that requires oxygen or another gas to be present, for example, as a reactant. As liquid is evaporated from chamber 110 through flexible member 114, the concentration of reactant and/or product within chamber 110 can increase. This increase in concentration of the reactant may be undesirable in some embodiments, such as when chamber 110 is being used as part of a chemostat. In some embodiments, after at least a portion of the liquid within chamber 110 has exited the chamber through a gas-permeable medium, a second liquid (e.g., additional liquid from inlet 116 or a second liquid from another liquid inlet) can be flowed into chamber 110 to increase the volume of liquid within the chamber, for example, after outlet 118 or another channel downstream of chamber 110 has been closed. In some embodiments, as additional liquid is transported into chamber 110, flexible member 114 can be deformed until it comes into contact with retaining structure 120. Flowing the second liquid into chamber 110 can have the effect of decreasing the concentration of a reactant within chamber 110. In some embodiments, transporting additional liquid into chamber 110 can cause the total volume of liquid within the chamber to increase by at least about 0.1%, at least about 1%, at least about 5%, at least about 10%, at least about 25%, at least about 50%, or at least about 100%.

In some embodiments, flexible member 114 is configured such that when a pressure is applied to chamber 110 (e.g., by adding a liquid to chamber 110 and/or by applying a vacuum to chamber 112) beyond a threshold level, flexible member 114 substantially conforms to the shape of retaining structure 120. This can be achieved, for example, by configuring retaining structure 120 to include a curved surface (e.g., a partial-spherical shape or other concave curved shape) that substantially matches the shape of flexible member 114 when it is deformed. FIG. 1B includes an exemplary cross-sectional schematic illustration of portion 100 when a liquid pressure is applied in chamber 110, causing deformation of flexible member 114. In FIG. 1B, when pressure is applied within chamber 110, and outlet 118 is closed, flexible member 114 substantially conforms to the shape of retaining structure 120. Because flexible member 114 cannot be deformed beyond the limits imposed by retaining structure 120 in this set of embodiments, the amount of liquid added to chamber 110 when outlet 118 is closed (or another portion of the system downstream of chamber 110 is closed) is substantially constant once a threshold pressure within chamber 110 is exceeded, regardless of the pressure of the liquid entering inlet 116. For example, in some embodiments, when liquid is transported into chamber 110 via inlet 116 at a relatively high pressure, retaining structure 120 can restrict flexible member 114 from deforming beyond the surface of structure 120, thereby defining a maximum volume of liquid that can be added to chamber 110 (i.e., the sum of the volumes of chambers 110 and 112 in FIG. 1A, as illustrated in FIG. 1B).

As used herein, a flexible member is said to “substantially conform” to the shape of an adjacent structure when the flexible member is deformed such that it contacts the adjacent structure essentially consistently along the flexible member. One of ordinary skill in the art would understand that, in this context, adjacent structures (e.g., a flexible member and a retaining structure) would not necessarily be in direct contact with each other, and that in some cases (e.g., as illustrated in FIG. 1A), adjacent structures include space between them. In some embodiments, when a flexible member substantially conforms to an adjacent structure such as a retaining structure, the volume between the retaining structure and the flexible member can be reduced by at least about 95%, at least about 99%, or at least about 99.9%, relative to the volume between the retaining structure and the flexible member when the flexible member is in an equilibrium state (i.e., when the pressure on each side of the flexible member is the same). For example, in the set of embodiments illustrated in FIGS. 1A and 1B, when chamber 110 is pressurized and flexible member 114 is deformed, the volume of chamber 112 can be decreased by at least about 95%, at least about 99%, or at least about 99.9% (as shown in FIG. 1B), relative to the volume of chamber 112 in its equilibrium state (as illustrated in FIG. 1A).

In some embodiments, when a flexible member substantially conforms to an adjacent structure such as a retaining structure, the volume between the flexible member and the retaining structure can be less than about 10%, less than about 5%, less than about 2%, or less than about 1% of the sum of the volume between the flexible member and the retaining structure and the volume between the flexible member and an adjacent chamber. For example, in the set of embodiments illustrated in FIGS. 1A-1C, flexible member 114 can be deformed toward retaining structure 120 such that the volume between flexible member 114 and retaining structure 120 is less than about 10%, less than about 5%, less than about 2%, or less than about 1% of the sum of the volumes of chambers 110 and 112.

In some embodiments, in addition to being deformed toward retaining structure 120, flexible member 114 can also be deformed toward wall 122 (or other suitable retaining structure) of chamber 110. For example, in some embodiments, flexible member 114 is configured to substantially conform to the shape of at least a portion of wall 122 of chamber 110 when a pressure within chamber 110 is less than a pressure outside chamber 110. In FIG. 1C, flexible member 114 has been deformed such that is substantially conforms to the shape of wall 122. This configuration can be achieved, for example, by pressurizing chamber 112 (e.g., beyond a threshold level). Pressurization of chamber 112 can be achieved, for example, by transporting a fluid (e.g., a gas such as oxygen, air, another gas, a liquid, or any other fluid) into chamber 112 via fluid inlet 124. Optionally, chamber 112 can also include a fluid outlet 126. In some embodiments in which chamber 112 includes outlet 126, pressurization of chamber 112 can be achieved by closing outlet 126 and flowing fluid into chamber 112 via inlet 124. In some such embodiments, constant pressure can be applied to chamber 112 via inlet 124, and outlet 126 can be controlled to either pressurize chamber 112 (by closing outlet 126) or vent chamber 112 (by opening outlet 126). In other embodiments, chamber 112 does not include an outlet, and, in some such embodiments, chamber 112 is pressurized by flowing fluid through inlet 124 and vented by stopping the flow of fluid through inlet 124.

Flexible member 114 can be made to substantially conform to wall 122 by adding a fluid to chamber 112 until the pressure within chamber 112 is sufficiently greater than the pressure within chamber 110 to cause flexible member 114 to conform to wall 122, as illustrated in FIG. 1C. The configuration illustrated in FIG. 1C can be useful, for example, when chambers 110 and 112 are being used as part of a pumping mechanism (e.g., a peristaltic pumping mechanism) and/or mixing mechanism, as described in more detail below.

In some embodiments, chamber 112 can be pressurized such that the volume between the walls of chamber 110 (i.e., wall 122 in FIGS. 1A-1C) and the flexible member can be reduced by at least about 95%, at least about 99%, or at least about 99.9%, relative to the volume between the walls of chamber 110 and the flexible member when the flexible member is in an equilibrium state (i.e., when the pressure on each side of the flexible member is the same). For example, in the set of embodiments illustrated in FIG. 1C, when chamber 112 is pressurized and flexible member 114 is deformed, the volume of chamber 110 can be decreased by at least about 95%, at least about 99%, or at least about 99.9%. In some embodiments, flexible member 114 can be deformed toward the walls of chamber 110 (i.e., wall 122 in FIG. 1C) such that the volume between flexible member 114 and the walls of chamber 110 is less than about 10%, less than about 5%, less than about 2%, or less than about 1% of the sum of the volumes of chambers 110 and 112.

In some embodiments, the flexible member and/or chambers can be configured such that the flexible member substantially conforms to the shape of retaining structure 120 and/or wall 122 at relatively low pressure differentials across the flexible member. For example, in some embodiments, flexible member 114 can be configured (e.g., by selecting suitable materials and/or dimensions for the flexible member) to substantially conform to the shape of retaining structure 120 when the difference between the pressure within chamber 110 and the pressure outside chamber 110 (e.g., within chamber 112) is at at least one value below 5 pounds per square inch (psi), at least one value below 3 psi, or at least one value below 1 psi. As one particular example, in some embodiments, the system is configured such that, when the pressure within chamber 112 is only 0.9 psi lower than the pressure within chamber 110, flexible member 114 substantially conforms to retaining structure 120.

In some embodiments, flexible member 114 can be configured (e.g., by selecting suitable materials and/or dimensions for the flexible member) to substantially conform to the shape of wall 122 when the difference between the pressure outside chamber 110 (e.g., within chamber 112) and the pressure inside chamber 110 is at at least one value below 5 pounds per square inch (psi), at least one value below 3 psi, or at least one value below 1 psi. As one particular example, in some embodiments, the system is configured such that, when the pressure within chamber 110 is only 0.9 psi lower than the pressure within chamber 112, flexible member 114 substantially conforms to wall 122.

The flexible member can be configured, in some embodiments, such that it does not undergo substantial plastic deformation during operation of the device. For example, in some embodiments, the flexible member can be configured such that, when it transitions from an unstrained state (e.g., as illustrated in FIG. 1A) to a state in which is substantially conforms to a retaining structure (e.g., as illustrated in FIG. 1B) and/or a chamber wall (e.g., as illustrated in FIG. 1C), it does not undergo substantial plastic deformation (e.g., it does not undergo any plastic deformation). This can be achieved, for example, by selecting suitable materials (e.g., elastic materials such as elastomers) and/or suitable dimensions for the flexible member.

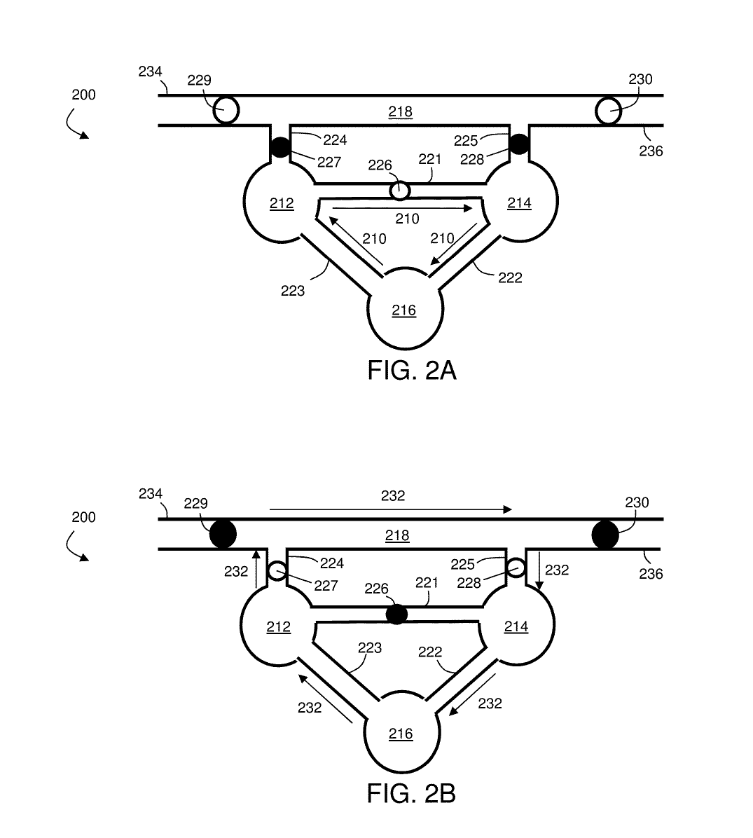

In some embodiments, multiple sets of chambers can be arranged such that fluidic mixing is achieved along one or more fluidic pathways. FIGS. 2A-2B include top-view schematic illustrations of a reactor 200 of a reactor system, according to one set of embodiments. In FIGS. 2A-2B, reactor 200 includes a first fluidic pathway indicated by arrows 210. The first fluidic pathway can include a first portion 212, a second portion 214, and a third portion 216. In some embodiments, one and/or more of (e.g., each of) portions 212, 214 and 216 corresponds to portion 100 illustrated in FIGS. 1A-1C, including the chambers, flexible members, and channel arrangements as illustrated and described. For example, in some embodiments, one and/or more of (e.g., each of) portions 212, 214, and 216 comprise a chamber similar to first chamber 110 in FIGS. 1A-1C, and flexible members within each chamber can be actuated to provide a peristaltic pumping mechanism that circulates liquid around first fluidic pathway 210 (e.g., from the chamber 110 within portion 212 to the chamber 110 within portion 214 to the chamber 110 within portion 216, or in the opposite direction), as described in more detail below. In some embodiments, one and/or more of (e.g., each of) portions 212, 214, and 216 comprise a retaining structure similar to retaining structure 120 in FIGS. 1A-1C and, in some cases, a second chamber similar to second chamber 112 in FIGS. 1A-1C.

In FIG. 2A, first portion 212 is fluidically connected to second portion 214 via channel 221. In addition, in FIG. 2A, portion 214 is connected to portion 216 via fluidic channel 222, and portion 216 is connected to portion 212 via fluidic channel 223. Reactor 200 can also include bypass channel 218. Bypass channel 218 can be fluidically connected to first portion 212 via channel 224 and second portion 214 via channel 225. In some embodiments, bypass channel 218 is substantially rigid. Bypass channel 218 can be configured (e.g., by selecting suitable materials and/or dimensions), in some embodiments, such that the volume of the bypass channel does not change by more than 2%, by more than 1%, or does not change at all during operation of the device.

Reactor 200 can also include a plurality of valves positioned in various places to control the flow of liquid within reactor 200. For example, channel 221 can be configured to include valve 226 between first portion 212 and second portion 214. In some embodiments, channel 225 is configured to include valve 228 positioned between second portion 214 and bypass channel 218. In some embodiments, channel 224 is configured to include valve 227 positioned between first portion 212 and bypass channel 218. In some embodiments, bypass channel 218 can include a first valve 229 positioned, for example, at an upstream location, and a second valve 230 positioned, for example, at a downstream location of the bypass channel.

Valves 226, 227, 228, 229, and 230 can be configured to control the flow of liquid within reactor 200. In some embodiments, valves 227 and 228 can be closed, and valve 226 can be open such that when portions 212, 214 and 216 are actuated, liquid is circulated along first continuous fluidic pathway 210. As illustrated in FIGS. 2A-2B, fluidic pathway 210 is arranged as a continuous loop. In some such embodiments, valves 227 and 228 can be opened such that a second fluidic pathway is formed including first portion 212, second portion 214, third portion 216, and bypass channel 218, as illustrated by arrows 232 in FIGS. 2A-2C. Optionally, in some such embodiments, valve 226 can be closed, thereby cutting off liquid flow between first portion 212 and second portion 214. In some such embodiments, valves 229 and 230 can be closed to ensure that the liquid remains within fluidic pathway 232. As illustrated in FIGS. 2A-2B, fluidic pathway 232 is arranged as a continuous loop. Valve 226 is an optional component, and in some embodiments, valve 226 is not present in the system. In some such embodiments, the second fluidic pathway formed includes portions 212, 214, 216, bypass channel 218, and channel 221 (including both pathways 210 and 232).

As noted elsewhere, in some embodiments, first portion 212, second portion 214, and third portion 216 can be actuated to transport liquid along fluidic pathway 210 and/or fluidic pathway 232. This can be achieved, for example, by configuring reactor 200 such that each of portions 212, 214, and 216 include the multi-chamber arrangements illustrated in FIGS. 1A-1C. In some embodiments, each of portions 212, 214, and 216 can be configured such that they are each able to assume a closed position wherein the flexible member 114 is strained to substantially conform to wall 122 of chamber 110, as illustrated, for example, in FIG. 1C. Portions 212, 214, and 216 can assume a closed position, for example, by transporting a gas is into chamber 112 via inlet 124. In some embodiments, each of portions 212, 214, and 216 can be configured such that they are each able to assume an open position wherein the flexible member 114 does not conform to wall 122, and liquid can be transported from inlet 116 to outlet 118, as illustrated in FIGS. 1A-1B.

Peristaltic mixing can be achieved by actuating first portion 212, second portion 214, and third portion 216 such that their operating states alternate between open (FIGS. 1A-1B) and closed (FIG. 1C) configurations. In some embodiments, three patterns may be employed to achieve peristaltic pumping: a first pattern in which first portion 212 and second portion 214 are closed and third portion 216 is open; a second pattern in which first portion 212 and third portion 216 are closed and second portion 214 is open; and a third pattern in which second portion 214 and third portion 216 are closed and first portion 212 is open. By transitioning among these three patterns (e.g., changing from the first pattern to the second pattern, from the second pattern to the third pattern, and from the third pattern to the first pattern, etc.) liquid can be transported among portions 212, 214, and 216 in a counter-clockwise direction (as illustrated in FIGS. 2A-2B). Of course, by re-arranging the order in which the patterns occur (e.g., by changing from the first pattern to the third pattern, from the third pattern to the second pattern, and from the second pattern to the first pattern, etc.), liquid can be transported in the clockwise direction as well. In some embodiments, first portion 212, second portion 214, and third portion 216 can be configured such that they cycle at a frequency of between about 0.1 Hertz and about 1000 Hertz, between about 0.5 Hertz and about 10 Hertz or between about 1 Hertz and about 3 Hertz. A cycle, in this context, refers to the time it takes for the flexible members within the loop to complete one pass through the series of patterns used to circulated the liquid. For example, in the system illustrated in FIGS. 2A-2B, a cycle corresponds to the time it takes to change from the first pattern to the second pattern to the third pattern and back to the first pattern, as described above. The frequency can then be calculated as the inverse of the time per cycle. In some embodiments, the frequency can be supplied by a computer, which can be used to program the opening and/or closing of portions 212, 214, and 216 within the system.

In FIGS. 2A-2B, when valves 227 and 228 are closed and valve 226 is open, liquid transport can be produced along fluidic pathway 210, as illustrated in FIG. 2A. When valves 227 and 228 are open and valves 229 and 230 are closed (and optionally, valve 226 is closed), liquid transport can be produced along fluidic pathway 232, as illustrated in FIG. 2B. The ability to switch between a configuration in which liquid is circulated within fluidic pathway 210 only and a configuration in which liquid is circulated within fluidic pathway 232 (in addition to or in place of circulation along pathway 210) can allow one to introduce fresh liquid (which can contain fresh reactant) into the reactor 200 (e.g., into pathway 210) without stopping operation of the device. For example, in one set of embodiments, valves 227 and 228 can be closed and valves 229 and 230 can be opened, after which liquid (which can contain fresh reactant) can be transported into bypass channel 218 via an upstream portion 234 of bypass channel 218. In some embodiments, some of the contents of portions 212, 214, and/or 216 may have been transported into bypass channel 218 prior to closing valves 227 and 228 (e.g., via pathway 232). In some such embodiments, the chamber contents that have been transported to bypass channel 218 after closing valves 227 and 228 can be transported out of bypass channel 218 and out of reactor 200 (e.g., via outlet 236) as liquid is transported into bypass channel 218. For example, in the set of embodiments illustrated in FIGS. 2A-2B, when bypass channel 218 is substantially rigid and valves 227 and 228 are closed, transportation of liquid into the bypass channel 218 from channel 234 results in substantially the same quantity of liquid transported out of bypass channel 218 via channel 236, which can serve to flush the bypass channel

Once fresh liquid has been transported into bypass channel 218, valves 226, 229, and 230 can be closed, and liquid (including the liquid located within pathway 210 as well as the fresh liquid within bypass channel 218) can be circulated along fluidic pathway 232 by actuating portions 212, 214, and 216. In some embodiments, once liquid has been circulated along fluidic pathway 232 for a certain period of time, valves 227 and 228 can be closed (and valve 226 can be opened if necessary), and portions 212, 214, and 216 can be actuated to circulate liquid along fluidic pathway 210.

In some embodiments, reactor 200 can be configured such that a chemical reaction takes place in the liquid within fluidic pathway 210 (and/or fluidic pathway 232) during mixing. For example, cell culture or another chemical or biochemical reaction can take place within fluidic pathway 210 (and/or fluidic pathway 232) while fluid is being transported. In some embodiments, one and/or more of first portion 212, second portion 214, and third portion 216 can include a gas-permeable medium (e.g., as part of the flexible member 114 described in FIGS. 1A-1C) configured to allow a gas to be transported into continuous pathway 210 (and/or continuous pathway 232) during the reaction. When configured in this fashion, flexible member(s) 114 can act as both a peristaltic pumping actuation mechanism as well as a pathway for gas entry into the reaction system.

In some embodiments, after a chemical and/or biological reaction has taken place within portions 212, 214, and/or 216 along fluidic pathway 210, a portion of the liquid can be removed from the fluidic pathway 210, for example, by opening valve 227 and/or 228. For example, in some embodiments, after a reaction has occurred within fluidic pathway 210, valve 226 can be closed and valves 227 and 228 can be opened, in some cases while the peristaltic mixing mechanism of portions 212, 214, and 216 is maintained. In some such embodiments, the liquid stored within bypass channel 218 can become part of the circulated fluid. After the liquid (e.g., including the liquid originally circulated along pathway 210 as well as the liquid contained within bypass channel 218) has been allowed to mix for a certain period of time (which can be pre-determined or variable), valves 227 and 228 can be closed and bypass channel 218 can be flushed (or the liquid can be partially replaced), for example by opening valves 229 and/or 230 and flowing fluid into the bypass channel (e.g., using an external pump or pressurized liquid). When operated in this fashion, a portion of the liquid that was originally circulated along pathway 210 is removed from pathway 210 (e.g., via outlet portion 236 of bypass channel 218), while continuous mixing and operation of the reaction system is maintained. The ability to operate the reactor in this fashion can allow one to continuously grow cells or perform other chemical or biochemical reactions, remove a portion of the liquid (e.g., containing a cell product, a chemical reaction product, and/or a biochemical reaction product), flush the removed liquid (e.g., for analysis or harvesting), and, in some cases, introduce new liquid (e.g., including fresh culture liquid, liquid containing additional reactant, or any other suitable liquid) into the reactor via bypass channel 218 while maintaining mixing and gas transfer.

In one exemplary operation configuration, fresh liquid can be introduced to the circulating reactor system by, for example, closing valves 227 and 228. Subsequently, valves 229 and 230 can be opened, and bypass channel 218 can be optionally flushed. In some embodiments, fresh liquid can be loaded into bypass channel 218. In some cases, valves 229 and 230 can then be closed and valves 227 and 228 can be opened (while valve 226 is closed) to switch from circulating liquid along pathway 210 to circulating liquid along pathway 232.

As noted elsewhere, in some cases, liquid can be transferred from the liquid flow stream within reactor 200 across, for example, a gas-permeable medium. For example, liquid within chambers 110 of portions 212, 214, and/or 216 can be transported (e.g., via evaporation) across the flexible members 114 within portions 212, 214 and/or 216. In some embodiments, after liquid has been transferred across a gas-permeable medium, the volume of liquid within one or more portions 212, 214, and/or 216 can be increased by flowing additional liquid into the reactor (e.g., into portions 212, 214, and/or 216). Transporting addition liquid into the reactor can cause the flexible member(s) within the portion(s) to be deflected when the appropriate valves are closed (e.g., outlet valve 230 and/or other valves). In some embodiments, transporting additional liquid into a reactor can cause the total volume of liquid within a chamber of the reactor (e.g., chambers 110 within portion 212, 214, and/or 216) to increase by at least about 0.1%, at least about 1%, at least about 5%, at least about 10%, at least about 25%, at least about 50%, or at least about 100%. In some embodiments, transporting additional liquid into a reactor portion can cause the total volume of liquid within the reactor to increase by at least about 0.1%, at least about 1%, at least about 5%, at least about 10%, at least about 25%, at least about 50%, or at least about 100%.

In some embodiments, the liquid that is transferred across a gas-permeable medium within a reactor portion can originate from a first inlet, while the additional liquid transported into that reactor portion can originate from a second, different inlet. For example, in the set of embodiments illustrated in FIGS. 2A-2B, a first liquid can be circulated along fluidic pathway 210 in a clockwise fashion, during which some of the first liquid can be evaporated from the system via a gas-permeable medium within portion 214. Subsequently, valve 226 can be closed, and a second liquid can be added to chamber 214 via channel 225. In some embodiments, valve 230 can be closed such that, as liquid is added to portion 214, the liquid volume within portion 214 increases as the flexible member within portion 214 is deflected.

In some embodiments, the liquid that is transferred across a gas-permeable medium within a reactor portion can originate from a first inlet, while the additional liquid transported into that reactor portion can originate from the same inlet. For example, in the set of embodiments illustrated in FIGS. 2A-2B, a first liquid can be circulated along fluidic pathway 210 in a clockwise fashion, during which some of the first portion of the liquid can be evaporated from the system via a gas-permeable medium within portion 216. Subsequently, valve 228 can be opened, and a second liquid originating from bypass channel 218 can be added to chamber 216 via channel 222.

In some embodiments, a portion of the additional liquid added to a reactor portion can also be transported out of the portion via the gas-permeable medium. The volume of liquid within the portion can be increased a second time by flowing additional liquid (e.g., a third liquid in the illustrative examples described above) into the reactor, causing the flexible member within the portion(s) to be deflected again. In some embodiments, the re-filling process described above can be repeated at least 1, 2, 3, 5, 10, 25, or 100 times or more during operation of the reactor. In addition, the total liquid volume within portion(s) 212, 214, and/or 216 can be substantially the same after each of the filling steps, in some cases (e.g., due to the flexible member substantially conforming to a retaining structure). For example, in some embodiments, transporting a second liquid into the chamber increases the liquid volume to a first volume, and transporting a third liquid into the chamber increases the liquid volume to a second volume that is substantially the same as the first volume.

In some embodiments, the components of reactor 200 (such as portions 212, 214, and 216) can be configured to reset the total volume of liquid within the reactor 200 to a substantially fixed amount (i.e., reactor 200 can undergo a liquid volume reset step). This can be achieved, for example, by closing valve 230 and flowing a liquid through inlet portion 234 of bypass channel 218. As the liquid enters bypass channel 218, it can also fill portions 212, 214, and/or 216; channels 221, 222, and 223 connecting portions 212, 214, and 216; and channels 224 and 225 connecting portions 212 and 214 to bypass channel 218. In some embodiments, one or more of portions 212, 214, and 216 can be closed (e.g., by pressurizing upper chamber 112 with a gas and conforming flexible member 114 to chamber wall 122, thereby removing the volume of liquid in chamber 110), which reduces the total amount of liquid that is transported into reactor 200.

In some embodiments, only one of portions 212, 214 and 216 is closed during the liquid volume reset step. For example, in some embodiments, second portion 216 is closed by pressurizing the upper chamber 112 of portion 216. Valve 230 can be closed, and liquid can be transported through inlet portion 234 of bypass channel 218 and into chambers 110 of portions 212 and 214. In some embodiments, as the liquid is transported into portions 212 and 214, the total amount of liquid added to the system is limited by the extent to which the flexible members 114 in each of portions 212 and 214 can be deflected. As described in relation to FIGS. 1A-1C, flexible members 114 within portions 212 and 214 can be deformed, in some embodiments, only until they come into contact with retaining structure 120. Thus, in some such embodiments, the total amount of liquid that is added to reactor 200 in the set of embodiments in which portion 216 is in a closed state corresponds to the sum of the volumes of chambers 110 and 112 in portions 212 and 214; the volumes of channels 221, 222, and 223; the volumes of channels 224 and 225; and the volume of bypass channel 218. In some embodiments, once liquid has been introduced into reactor 200 to reset the total amount of liquid to a fixed volume, circulation of the liquid is resumed. For example, valves 227 and 228 can be closed, valve 226 can be opened (if necessary), and circulation of liquid can be resumed along pathway 210. In some embodiments, valves 229 and 230 can be closed, valves 227 and 228 can be opened, and valve 226 can be closed (if necessary) to circulate liquid along fluidic pathway 232.

In some embodiments, multiple liquid volume reset steps can be performed on reactor 200. For example, after some amount of time (which can be pre-determined or variable) has passed since the last liquid volume reset step, peristaltic mixing can be halted, and the total volume of liquid within reactor 200 can be reset according to the liquid volume reset procedure outlined above. In some embodiments, by resetting the total volume of liquid within reactor 200 after certain periods of time, reactor 200 can simulate the performance of a constant volume reactor, which can be desirable for chemostat or turbidostat operation.

While the liquid volume reset step has been primarily described as involving the closure of one of portions 212, 214, and 216 (e.g., by pressurizing chamber 112 within the portion), it should be understood that in other embodiments, two of portions 212, 214, and 216 can be closed during the liquid volume reset step. In addition, while the set of embodiments illustrated in FIGS. 2A-2B include three portions 212, 214, and 216 (one and/or more of (e.g., all of) which can correspond to portion 100 as illustrated in FIGS. 1A-1C), it should be understood that, in other embodiments, additional pathway portions (one and/or more of (e.g., all of) which can correspond to portion 100 as illustrated in FIGS. 1A-1C) can be included within reactor 200. For example, in some embodiments, at least 4, at least 5, at least 10, at least 25, at least 50 or more portions (which can each include chambers 110 and 112 as illustrated in FIGS. 1A-1C) can be included in a continuous fluid pathway such as reactor 200. In some embodiments, more than one bypass channel can be employed, for example, to remove the reaction products produced in reactor 200.

In addition to providing a peristaltic pumping mechanism and allowing the addition of a constant volume of liquid to the reaction portion during the liquid volume reset step, the chambers illustrated in FIGS. 1A-1C can also be used to introduce liquid at a substantially fixed pressure relatively easily and without the use of expensive equipment. Referring again to FIGS. 1A-1C, flexible member 114 can be used as a peristaltic pump to provide liquid through outlet 118 at a substantially constant pressure. In one set of embodiments, a valve within outlet 118 can be closed while liquid is transported into chamber 110 via inlet 116. Once chamber 110 has been filled, as illustrated in FIG. 1B, a valve in inlet 116 can be closed. To deliver the liquid within chamber 110 at a constant pressure, the valve within outlet 118 can be opened and a gas can be transported through inlet 124 to depress flexible member 114 at a controlled rate. Eventually, flexible member 114 can assume the configuration illustrated in FIG. 1A, and if allowed to proceed further, can assume the configuration illustrated in FIG. 1C. After a period of time, the valve in outlet 118 can be closed, the valve in inlet 116 can be opened, and additional liquid can be transported into chamber 110. The process outlined above can be repeated to deliver the new liquid within chamber 110 at a substantially constant pressure. In some embodiments, two portions 100 (or more) can be operated in an alternate fashion such that liquid is being supplied at a constant pressure continuously without the need for stopping the downstream process that requires the liquid. The mechanism outlined above can be used to provide liquid, for example, to bypass channel 218 illustrated in FIGS. 2A-2C.

Transporting liquid at a substantially fixed pressure using the chambers illustrated in FIGS. 1A-1C can be relatively easy and inexpensive because it is generally easier to control the upstream pressure of a gas stream than it is to control the upstream pressure of a liquid stream. For example, the upstream pressure of a gas stream can usually be controlled using relatively inexpensive equipment such as a gas flow regulator. On the other hand, controlling the pressure of an upstream liquid stream can require the use of relatively expensive equipment such as a syringe pump. In some such embodiments in which portion 100 is used to transport a liquid at a substantially fixed pressure, the pressure within inlet 116 does not need to be controlled because the amount of liquid added to chamber 110 via inlet 116 will be limited by the extent to which flexible member 114 can be deformed (which, in FIGS. 1A-1C, is limited by retaining structure 120). Once the valve within inlet 116 has been closed, the pressure within chamber 112 (which can be relatively easily controlled using, for example, a gas flow regulator) will determine the pressure drop from chamber 110 to downstream locations (e.g., outlet 118 and other downstream locations).

In some embodiments (e.g., in which a portion 100 is used to transport a liquid at a substantially fixed pressure), the stress applied by the liquid within chamber 110 on flexible member 114 (which can result in flexible member substantially conforming to retaining structure 120) can be smaller than the pressure within chamber 112 when chamber 112 is subsequently used to drive liquid flow out of chamber 110 via outlet 118. For example, in some embodiments, the stress applied by the liquid within chamber 110 on flexible member 114 can be at least about 5%, at least about 10%, at least about 20%, or at least about 50% smaller than the pressure within chamber 112 when chamber 112 is subsequently used to drive liquid flow out of chamber 110 via outlet 118, measured relative to the pressure within chamber 112 during liquid removal. In some cases, if the stress applied by the liquid within chamber 110 on flexible member 114 is similar to or greater than the pressure within chamber 112 when chamber 112 is subsequently used to drive liquid flow out of chamber 110. the hydraulic pressure from inlet 116 can be translated into membrane stress, which can be translated into hydraulic pressure at the output 118 when a valve within inlet 116 is closed.

In some embodiments (e.g., in which a portion 100 is used to transport a liquid at a substantially fixed pressure), the stress applied by the liquid within chamber 110 on flexible member 114 can be much smaller than the pressure within chamber 112 when chamber 112 is subsequently used to drive liquid flow out of chamber 110 via outlet 118. In some such cases, pressurizing chamber 112 can be used to deliver liquid at a constant pressure regardless of whether flexible member 114 contact (e.g., substantially conforms to) retaining structure 120 prior to pressurizing chamber 112.

As noted above, the systems and devices described herein (e.g., chamber 110, reactor 200, etc.) can comprise one or more cells which can be used, for example, in part of a cell culture process. As used herein, a “cell” is given its ordinary meaning as used in biology. One or more cells and/or one or more cell types can be contained in a the systems and devices described herein (e.g., in a chamber, a channel, the reactor portion, etc.). The cell may be any cell or cell type. For example, the cell may be a bacterium (e.g., E. coli) or other single-cell organism, a plant cell, or an animal cell. If the cell is a single-cell organism, then the cell may be, for example, a protozoan, a trypanosome, an amoeba, a yeast cell, algae, etc. If the cell is an animal cell, the cell may be, for example, an invertebrate cell (e.g., a cell from a fruit fly), a fish cell (e.g., a zebrafish cell), an amphibian cell (e.g., a frog cell), a reptile cell, a bird cell, or a mammalian cell such as a primate cell, a bovine cell, a horse cell, a porcine cell, a goat cell, a dog cell, a cat cell, or a cell from a rodent such as a rat or a mouse. In some embodiments, the cell can be a human cell. In some embodiments, the cell may be a hamster cell, such as a Chinese hamster ovary (CHO) cell. If the cell is from a multicellular organism, the cell may be from any part of the organism. For instance, if the cell is from an animal, the cell may be a cardiac cell, a fibroblast, a keratinocyte, a heptaocyte, a chondracyte, a neural cell, a osteocyte, a muscle cell, a blood cell, an endothelial cell, an immune cell (e.g., a T-cell, a B-cell, a macrophage, a neutrophil, a basophil, a mast cell, an eosinophil), a stem cell, etc. In some cases, the cell may be a genetically engineered cell. In some embodiments, the liquid transported into reactor 200 (e.g., into one or more portions 100) within the device can comprise a culture medium suitable for use with any of the cells described herein. It should be understood that cell culture includes the growth of any cell type, including specific cell types derived from plants or animals as well as the growth of microorganisms (e.g., bacteria, yeast, etc.).

In some embodiments in which cell culture is performed, the liquid transported into chamber 110 and/or circulated within reactor 200 can comprise a cell culture medium which can contain one or more reactants suitable for use in cell culture. Suitable reactants for use in cell culture systems include any materials that can be at least partially metabolized by a cell, e.g., to produce one or more metabolites from the cell. Exemplary reactants include, for example, sugars (e.g., xylose, deoxyribose, sucrose, fructose, glucose, galactose, etc.) or other suitable carbohydrates; amino acids (e.g., aspartic acid, lysine, etc.); nucleic acids (e.g., RNA, siRNA, RNAi, DNA, PNA, etc.); and/or other species such as proteins, peptides, enzymes, etc. In some embodiments, the reactant can comprise an inhibitor, a radiation source, a growth factor. In some embodiments, the reactant may not have a direct impact as a metabolite. One of ordinary skill in the art would be capable of selecting a suitable cell culture medium based upon the type of cell being cultured within reactor 200.

In some embodiments, portion(s) 100 and/or reactor 200 can be part of a system that can interface with outside equipment. For example, in some embodiments, portion(s) 100 and/or reactor 200 may be configured to interface with microfluidic/microscale equipment. In some embodiments, the devices described herein can be configured to interface with macroscopic equipment in addition to or in place of microfluidic/microscale equipment.

The devices described herein can comprise, in some embodiments, one or more sensors. For example, the device can include sensors that monitor a gas and/or liquid within a chamber of reactor 200, a component within a channel connected (directly or indirectly) to reactor 200, and/or the substrate(s) of the device. The sensors within the device can be configured to determine temperature, pressure, flow rates, pH, chemical concentration, dissolved oxygen, turbidity, and/or other properties.

In some embodiments, the devices described herein can include one or more control elements. For example, the temperature of a component can be controlled using heat exchangers, for example, in contact with the substrate in which the chamber resides. pH can be controlled by the addition of chemicals. Dissolved oxygen levels can be controlled by adjusting the flow of oxygen into a channel or chamber. Computerized control systems can be used, in some embodiments, to monitor and control the operation of the device.

The devices described herein can be fabricated from a variety of materials and using a variety of methods. For example, referring back to FIGS. 1A-1C, chambers 110 and 112, channels 116 and 118, and channels 124 and 126 can be formed in one or more substrates (e.g., substrates 160 and 162). In addition, in FIGS. 2A-2B, portions 212, 214, and 216; channels 218, 224, 225, 221, 222, and 223; and other portions of the reactor portion can be formed in one or more substrates. Suitable substrate materials include, but are not limited to polymers (e.g., polyethylene, polystyrene, polycarbonate, poly(dimethylsiloxane) (PDMS), poly(methyl methacrylate) (PMMA), polytetrafluoroethylene (PTFE), a cyclo-olefin copolymer (COC), a cyclo-olefin polymer (COP)), glass, quartz, and silicon. Those of ordinary skill in the art, given the present disclosure, can readily select a suitable substrate material based upon e.g., its rigidity, its inertness to (e.g., freedom from degradation by) a fluid to be passed through it, its robustness at a temperature at which a particular device is to be used, and/or its transparency/opacity to light (e.g., in the ultraviolet and visible regions). In some instances, substrates 160 and 162 can be formed of the same material. In other cases, substrates 160 and 162 can be formed of different materials.

The chambers and channels described in FIGS. 1A-1C and 2A-2B can be formed, for example, using etching, milling, soft lithography, embossing, injection molding, or any other techniques compatible with channel fabrication.

Flexible member 114 can also be formed of a variety of materials. In some embodiments, all or part of flexible member 114 can be formed of a polymeric material. In some embodiments, flexible member 114 can comprise an elastomeric material (i.e., an elastic polymer), for example, having a Young's modulus of less than about 1 GPa. A variety of elastomeric polymeric materials are suitable for making flexible member 114 including, for example, polymers of the general classes of silicone polymers, epoxy polymers, and acrylate polymers. Epoxy polymers are characterized by the presence of a three-member cyclic ether group commonly referred to as an epoxy group, 1, 2-epoxide, or oxirane. As specific examples, diglycidyl ethers of bisphenol A may be used, in addition to compounds based on aromatic amine, triazine, and cycloaliphatic backbones. Other examples include the well-known Novolac polymers, silicone elastomers formed from precursors including the chlorosilanes such as methylchlorosilanes, ethylchlorosilanes, and phenylchlorosilanes, and the like. In some embodiments, flexible member 114 comprises polydimethylsiloxane (PDMS). Exemplary polydimethylsiloxane polymers include those sold under the trademark Sylgard by the Dow Chemical Company, Midland Mich., and particularly Sylgard 182, Sylgard 184, and Sylgard 186. In some embodiments, flexible member 114 can comprise a poly(p-xylylene) polymer (e.g., a parylene), a thiolene, a polyurethane, a fluoropolymer (e.g., Teflon®), and/or latex.

In some embodiments, flexible member 114 can comprise a gas-permeable material. The use of gas-permeable materials in flexible member 114 can allow chamber 112 to be configured as both a liquid flow actuator as well as a gas-delivery vessel (e.g., when oxygen or other gases are required for a reaction performed in chamber 110). Exemplary gas-permeable materials that can be used to make flexible member 114 include, but are not limited to, silicon-based polymers such as polydimethylsiloxane (PDMS) (including any of the PDMS polymers mentioned elsewhere herein), fluorinated ethylene propylene (FEP), perfluoroalkoxy (PFA), polytetrafluoroethylene (PTFE), polyurethane, and/or thiolene.

As noted elsewhere, flexible member 114 or another portion(s) of a wall of chamber 110 and/or chamber 112 can, in certain embodiments, comprise a gas-permeable medium. In some embodiments, at least a portion of one and/or more walls of chamber 110 and/or chamber 112 (e.g., such as flexible member 114) can be formed of a material that has a permeability to oxygen gas (O₂) of at least about 10 Barrer, at least about 50 Barrer, at least about 100 Barrer, at least about 250 Barrer, at least about 500 Barrer, at least about 750 Barrer. In some embodiments, the flexible member can be formed of a material that has a permeability to oxygen gas (O₂) of less than about 5000 Barrer, less than about 1000 Barrer, or less than about 850 Barrer. One of ordinary skill in the art would recognize that 1 Barrer is equal to 10⁻¹⁰ cm³ (STP) cm/(cm² s cmHg), wherein STP refers to standard temperature and pressure (i.e., referring to a temperature of 273.15 K (0° C.) and a pressure of 101,325 Pa (1 atmosphere). One or ordinary skill in the art would be capable of determining the oxygen permeability of a material using, for example, the standard method defined by the American Society of Testing and Materials D3985 standard (ASTM, 1995), which is incorporated herein by reference. In some embodiments, at least a portion of a wall of chamber 110 and/or chamber 112 (e.g., such as flexible member 114) can be formed of a material that is permeable to carbon dioxide.

In some embodiments, the reactor can exhibit a relatively high oxygen transfer rate (k_{L}a). For example, in some embodiments, the reactor can exhibit an oxygen transfer rate of at least about 0.1 hours⁻¹, at least about 1 hours⁻¹, at least about 10 hours⁻¹, at least about 50 hours⁻¹, between about 0.1 hours⁻¹ and about 100 hours⁻¹, between about 1 hours⁻¹ and about 100 hours⁻¹, between about 10 hours⁻¹, and about 100 hours⁻¹ at an input pressure of 3 psi. One of ordinary skill in the art would be capable of determining the oxygen transfer rate of a system using the dynamic gassing method as described, for example, in V. Linek, P. Benes, and V. Vacek, “Measurement of aeration capacity of fermenters,” Chem. Eng. Technol., 1989, Vol. 12, Issue 1, pages 213-217, which is incorporated herein by reference.

In some embodiments, flexible member 114 can be formed as a thin film. For example, in some embodiments, flexible member 114 may have an average thickness of less than about 1 mm, less than about 100 micrometers, or less than about 50 micrometers. In some embodiments, the average thickness of flexible member 114 can be at least about 100 nm or at least about 1 micrometer. One of ordinary skill in the art would be capable of measuring the average thickness of a thin film, for example, via an optical coherence interferometer. Flexible member 114 can be formed as a thin film by, for example, spray coating or spin coating a material (e.g., a monomeric precursor material, a liquefied precursor, etc.) onto a substrate to achieve the desired thickness.

The channels described herein can have any cross-sectional shape (circular, semi-circular, oval, semi-oval, triangular, irregular, square or rectangular, or the like) and can be covered or uncovered. In embodiments where a channel is completely covered, at least one portion of the channel can have a cross-section that is completely enclosed, or the entire channel may be completely enclosed along its entire length with the exception of its inlet(s) and outlet(s). A channel (including a microfluidic channel) may also have an aspect ratio (length to average cross sectional dimension) of at least 0.5:1, at least 1:1, at least 2:1, at least 3:1, at least 5:1, or at least 10:1. An open or partially open channel, if present, may include characteristics that facilitate control over fluid transport, e.g., structural characteristics (an elongated indentation) and/or physical or chemical characteristics (hydrophobicity vs. hydrophilicity) or other characteristics that can exert a force (e.g., a containing force) on a fluid. The fluid within the channel may partially or completely fill the channel. In some cases where an open channel is used, the fluid may be held within the channel, for example, using surface tension (e.g., a concave or convex meniscus).

The systems described herein may be microfluidic, in some embodiments, although the invention is not limited to microfluidic systems and may relate to other types of fluidic systems. “Microfluidic,” as used herein, refers to a device, apparatus or system including at least one fluid channel having a cross-sectional dimension of less than about 1 mm. A “microfluidic channel,” as used herein, is a channel meeting these criteria. The “cross-sectional dimension” (e.g., a diameter) of the channel is measured perpendicular to the direction of fluid flow. In some embodiments, the devices described herein include at least one channel having a maximum cross-sectional dimension of less than about 500 micrometers, less than 200 micrometers, less than about 100 micrometers, less than 50 micrometers, or less than about 25 micrometers.

In some embodiments, reactor 200 can be configured to contain relatively small volumes. For example, reactor 200 (including the sum of the volumes of fluidic pathways 210 and 232, including bypass channel 218; portions 212, 214, and 216 (and other portions if present); and channels 221, 222, 223, 224, and 225) can have a maximum total liquid volume (measured when flexible members, if present, substantially conform to retaining structures 120) of less than about 100 mL, less than about 10 mL, less than about 5 mL, less than about 1 mL, or less than about 100 μL. In some embodiments, reactor 200 can have a maximum total liquid volume of at least about 10 nL or at least about 100 nL. In some embodiments, the sum of the volume of pathway 210 and the volume of the bypass channel is less than about 100 mL, less than about 10 mL, less than about 5 mL, less than about 1 mL, or less than about 100 μL, and/or, in some embodiments, at least about 10 nL or at least about 100 nL.

In some cases, the dimensions of the channel may be chosen such that fluid is able to freely flow through the article or substrate. The dimensions of the channel may also be chosen, for example, to allow a certain volumetric or linear flow rate of fluid in the channel. Of course, the number of channels and the shape of the channels can be varied by any method known to those of ordinary skill in the art.

The chambers described herein can also have any suitable shape and/or size, cross-sectional or otherwise. In some embodiments, the chamber can be larger than one and/or more the conduits (e.g., inlets and/or outlets) to which it is connected, as illustrated in FIGS. 1A-1C. For example, the cross-sectional dimension of a chamber (e.g., measured perpendicular to fluid flow) can be larger (e.g., at least about 10%, at least about 25%, at least about 50%, at least about 100% larger, at least about 200% larger, at least about 400% larger, or at least 1600% larger) than the corresponding cross-sectional dimension of at least one fluidic conduit (e.g., inlet and/or outlet) connected to the chamber, measured relative to the cross-sectional dimension of the conduit.

The valves within the systems and devices described herein (e.g., valve 226, 227, 228, 229, and/or 230) can be formed using a variety of suitable methods. In some embodiments, the valves can comprise structures similar to those illustrated in FIGS. 1A-1C. In such embodiments, liquid flow can be stopped by pressurizing chamber 112 such that flexible member 114 conforms to wall 122, as illustrated in FIG. 1C. In other embodiments, other types of valves can be employed. One of ordinary skill in the art would be capable of selecting appropriate valves for use in the embodiments described herein.

The devices described herein can be formed, for example, by bonding flexible member 114 with other components (e.g., substrate material components in which the chambers and channels are formed) to form the assembled article. For example, in some embodiments, chambers and channels (or portions of chambers and channels) can be formed in a first substrate (e.g., substrate 160 in FIGS. 1A-1C) and additional chambers and channels (or portions of chambers and channels) can be formed in a second substrate (e.g., substrate 162 in FIGS. 1A-1C). In some embodiments, the flexible member can be positioned between the first and second substrates and bonded between them. For example, in FIGS. 1A-1C, flexible member 114 can be positioned between substrates 160 and 162 and bonded between them to form the assembled structures. In some embodiments, one or more substrates can be treated with a chemical to achieve the desired bond. In some embodiments, after initial assembly, the bonded components can be pressed and/or heated to achieve the final bond. Systems and methods for forming bonds between materials that can be used in the flexible member and the substrates described herein are described, for example, in U.S. Patent Publication No. 2011/0195260 to Lee et al., published on Aug. 11, 2011, and entitled “Method of Hydrolytically Stable Bonding of Elastomers to Substrates,” which is incorporated herein by reference in its entirety for all purposes. Of course, other techniques can also be used to form a seal between the substrate(s) and the flexible members described herein, including the use of adhesives, gluing, welding (e.g., ultrasonic), and/or mechanical methods (e.g., clamping).

Transporting liquids or other fluids within the systems and devices described herein can be achieved by any suitable method. For example, in some embodiments, a pressure gradient can be established by applying a positive pressure to the inlet of a channel using, for example, a pump, by use of gravity, or by any other suitable method. In some embodiments, pressure gradients within a channel can be established by applying a negative pressure to one end of a channel (e.g., an outlet of a channel), for example, via attachment of a vacuum pump to an outlet, withdrawal of air from a syringe attached to an outlet, or by any other suitable method. Fluid transport can also be achieved using peristaltic pumping configurations, including those described elsewhere herein.

The following examples are intended to illustrate certain embodiments of the present invention, but do not exemplify the full scope of the invention.

### Example 1

This example describes the fabrication and testing of a system for continuously culturing cells.

### Device Design

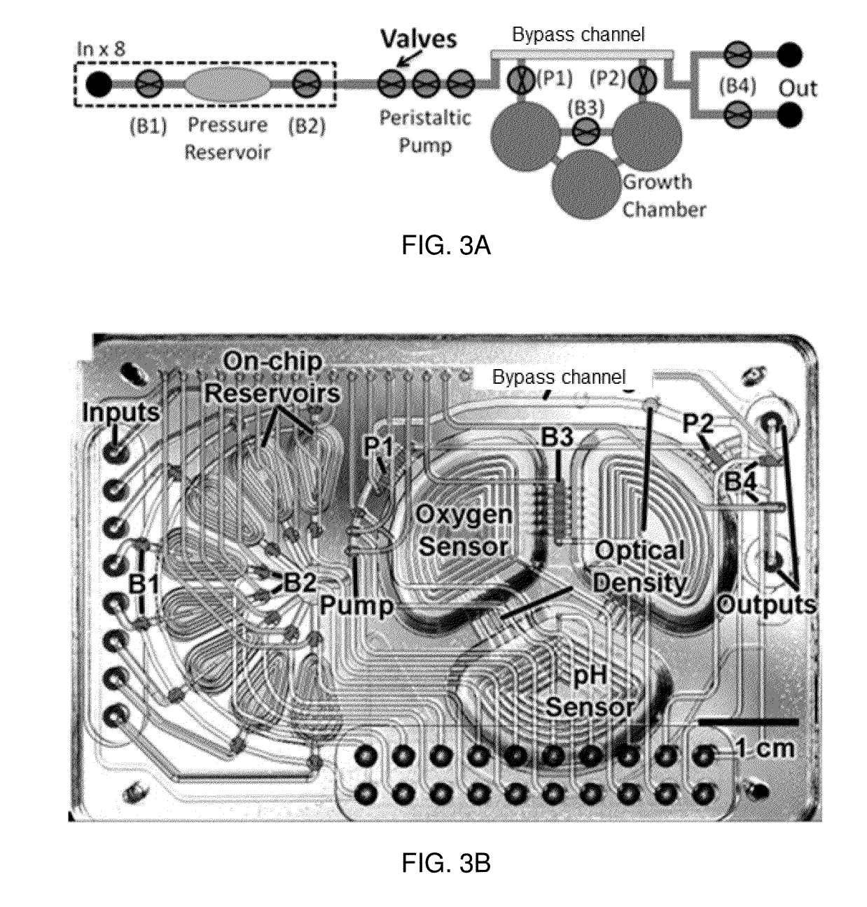

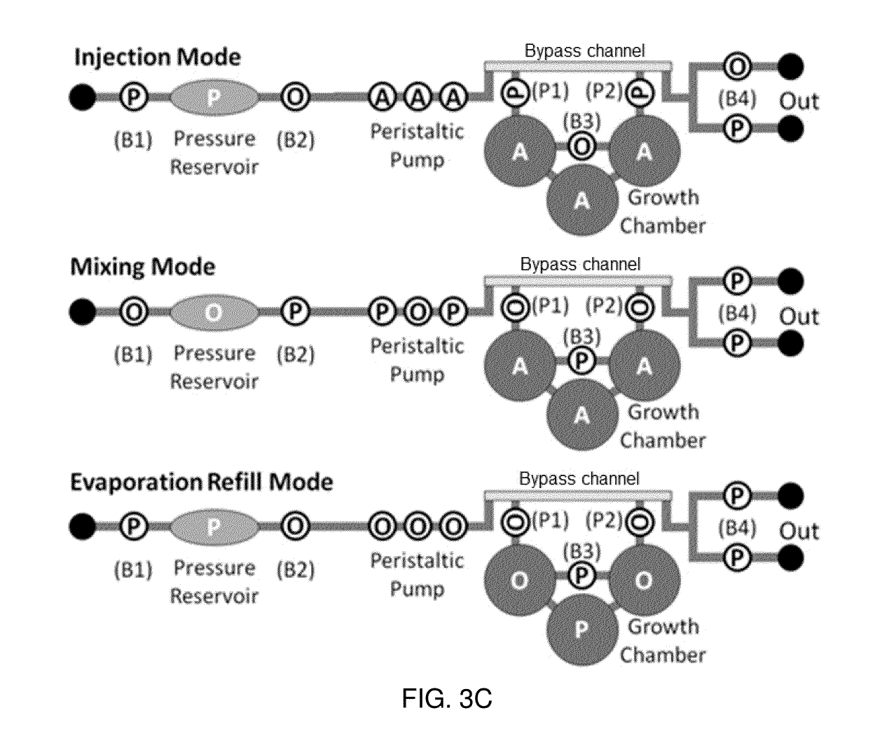

FIGS. 3A-3C are schematic illustrations and a photograph of the cell culture device. The continuous culture device had a 1 mL working volume. The following component descriptions are organized from input to output. Eight inputs for fluids were located at the top of the chip for different media and were connected to 35-μL on-chip reservoirs to reduce pressure variations. A single peristaltic pump with an injection volume of 210 nL was connected between all 8 reservoirs and the growth chambers (corresponding to portions 212, 214, and 216 in FIGS. 2A-2B). The three interconnected growth chamber sections contained flexible members made of PDMS membranes, and could be inflated to a total liquid volume of 500 μL per chamber. With a designed volume of 1 mL, only two sections were filled, allowing compliance for mixing. Two outputs were provided to enable automatic switching between sample collection and waste output. The connection between the peristaltic pump and outputs is labeled as the bypass channel (corresponding to channel 218 in FIGS. 2A-2B. The microfluidic bypass channel had a volume of 25 μL and was also connected in series with the mixer to allow mixing of newly injected media with the growth chamber contents.

Peristaltic Mixer:

The growth chamber also functioned as a peristaltic mixer and included three growth wells, each with symmetrically rounded 250-μL top and bottom halves separated by PDMS membranes. These wells acted as valves and could deflect from their equilibrium position to inflate and fill with liquid, or deflate to remove liquid. Active actuation of the three membranes in a circular pattern resulted in mixing through the interconnected channels. Mixing was achieved by operating the three chambers in a pattern of {PPO, POP, OPP}, where (P) indicates pressurized and (O) indicates open and vented. The pressurization state was changed every 333 ms during growth.

Peristaltic Pump:

Fluid injections were mediated by 8 separate 35-μL on-chip reservoirs and a single peristaltic pump with a nominal injection volume of 220 nL. The peristaltic pump consisted of 3 valves, which could move discrete plugs of fluid if actuated in the pattern {POP, POO, PPO, OPP, OOP}. The ceiling of the peristaltic pump center valve was designed to nearly equal the volume of the final valve, reducing the backward step typical of peristaltic pumps.

On-Chip Reservoir Isolation:

Blocking valves were used to enable fluid pressurization as well as prevent diffusion between different fluid sections of the device. The external fluid inputs were isolated from the on-chip reservoirs by individual blocking valves (B1). These valves mediated filling of the on-chip reservoirs. The on-chip reservoirs were also isolated from the peristaltic pump by individual blocking valves (B2). These prevented diffusion between different inputs and also allowed for pump input selection.

Bypass Channel:

The growth chamber had a variable volume due to the elasticity of the PDMS membranes, which were used for peristaltic mixing and valving. As a result, fluid pumped into the growth chamber could accumulate, changing the growth chamber volume. In addition, since the growth chamber was peristaltically mixed, it was under constant pressure resulting in complete removal of the growth chamber contents if a direct output connection was allowed. To mediate volume changes due to the peristaltic pump and growth chamber pressure, an additional constant volume microfluidic channel (designated as a “bypass channel”) was included, connecting the peristaltic pump directly to the output. As shown in FIG. 3A, the peristaltic pump and output were connected through two paths, one going through the growth chamber, and one going through the bypass channel. Two additional valves (P1, P2) were located at the input and output of the growth chamber which allowed for selection of the fluid path to include or exclude the growth chamber. A blocking valve (B3) was also located between two growth chamber sections, which could be used to force circular flow between the growth chamber and bypass channel. In addition, a blocking valve (B4) was placed before the output. The configuration of the bypass channel maintained the growth chamber volume.

### Device Operation

Maintaining growth chamber volume while providing integrated peristaltic mixing and flow control involved multiple operation steps mediated by the bypass channel. Three configurations were used, as shown in FIG. 3C: injection mode, mixing mode, and evaporation refill mode. In general, the chip was placed into either injection or mixing configurations depending on whether a pumping cycle needed to be initiated. At pre-programmed times, evaporation refill mode was initiated to return the chamber to full volume.

Injection Mode:

Fluid injection from a single input was performed using a specific valve configuration. The external input was closed (B1=P), the on-chip reservoirs were pressurized, and one reservoir was connected to the peristaltic pump (B2=O). This allowed the pressure from the on-chip reservoir to drive the peristaltic pump. The growth chamber was then disconnected from the rest of the device (P1=P and P2=P) and the bypass channel was directly connected to the output (B4=O). Growth chamber mixing was still enabled by opening the second mixer valve (B3=O). In this configuration, peristaltic pump injections resulted in new fluid entering the bypass channel while forcing old growth chamber fluid out of the device. This allowed extraction of growth chamber fluid as long as the newly injected volume was less than the total bypass channel volume and also ensured that the input and output flow rates were identical.

Mixing Mode:

Once injection into the bypass channel was complete, the chip was switched to mixing mode. The peristaltic pump was turned off and blocked, the output was closed (B4=P), and the on-chip reservoir connections were closed (B2=P). After closing the input and output valves, connecting the growth chamber to the bypass channel (P1=O and P2=O) while closing the second mixer valve (B3=P) forced the mixer to circulate through the bypass channel, mixing the new fluid contents with the growth chamber fluid. In addition, opening the external input blocking valve (B1=O) allowed the on-chip reservoir to refill through the external input fluid, setting up the on-chip reservoir for the next injection.

Evaporation Refill Mode:

In this mode, the growth chamber mixer was turned off and only one section was pressurized, as illustrated in FIG. 3C. This forced the growth chamber liquid into the two unpressurized sections. When evaporation occurred, the membranes did not inflate fully in the unpressurized chambers. After setting the growth chamber to the proper state, the external inputs were closed (B1=P), the on-chip reservoir was pressurized, and the water input valve was connected to the peristaltic pump (B2=O). All three peristaltic pump valves were then opened, allowing a direct connection between the pressurized on-chip reservoir and the growth chamber. The pressurized water from the reservoir reinflated the unpressurized mixer sections until the membranes are fixed against the rigid chamber wall, returning the volume to 1 mL. The on-chip reservoir was generally maintained at a pressure that was less than the growth chamber membrane pressure to allow for the water flow to stop when the two unpressurized sections were full. After evaporation compensation, the device was again placed into mixing mode.

### Device Fabrication

The continuous culture microdevices were fabricated out of polycarbonate (PC) and polydimethylsiloxane (PDMS). Microchannels and features were machined into polycarbonate using a CNC milling machine (Minitech Minimill 4) with various sized ball, square, drill, and keyseat end mills. After machining, devices were polished using methylene chloride vapor to remove tooling marks and increase optical clarity. To remove absorbed solvent, the parts were then annealed at 130° C. in a convection oven.

After polishing device layers, devices were etched in 3M sodium hydroxide solution and rinsed in isopropanol to activate the surface. Then a chemical adhesive (Bis-n-MO isopropoxy-m-methoxy-silyl-propylamine) was applied to the surface with a wiper to introduce silicon dioxide groups at the surface. After baking at 65° C. in high humidity to harden the coating, a 65-μm thick PDMS membrane was corona treated for 30 seconds using a corona treater (Electrotechnic Products B0-20AC) and bonded to the coated PC device. For layers without temperature sensitive components such as optical sensors, parts were subjected to a second thermal anneal at 130° C. to accelerate bonding. For fluid layers, optical sensors were secured in the base of the fluid chamber using double-sided silicone adhesive tape (AR-clad 7876).

PDMS membranes were fabricated by spin coating PDMS onto an anti-static polyester transparency (Polymex PRI72). Thicknesses were monitored on-line using an optical coherence interferometer. After coating the transparency with the desired thickness of PDMS, the PDMS film was baked at 65° C. for 4 hours. The coating and bonding process was then repeated with subsequent layers to form multilayer stacks of PC-PDMS-PC. Due to the tacky nature of the initial bond, finger pressure was enough to initiate bonding making hydraulic presses and vices unnecessary for the bonding process. After bonding, the two layer device was baked at 50° C. for 4 hours or left at room temperature overnight. For passive fluid and gas layers, silicone adhesive tape was used instead of chemical adhesive to reduce fabrication complexity. A full layer stack including four polycarbonate layers, one PDMS valving membrane, and two silicone adhesive layers was produced.

Finally, the internal surfaces of the devices were modified with PEG. Internal surfaces were first coated with a 5% aqueous solution of Amino-Ethyl-Amino-Propyl-Silanetriol (Gelest Inc.) for 12 hours at room temperature. After rinsing with DI water, the device was coated with a copolymer of polyethylene glycol (PEG) and polyacryJic acid (PAA) for 12 hours at room temperature. Synthesis of the PEG-PAA copolymer was performed by mixing polyethylcne glycol (PEG) with amine functionality (Surfonamine® L-300) and polyacrylic acid (molecular weight: 5000) (PAA) with a 50% grafting ratio and heating at 120° C. in a nitrogen environment.

### Continuous Culture System

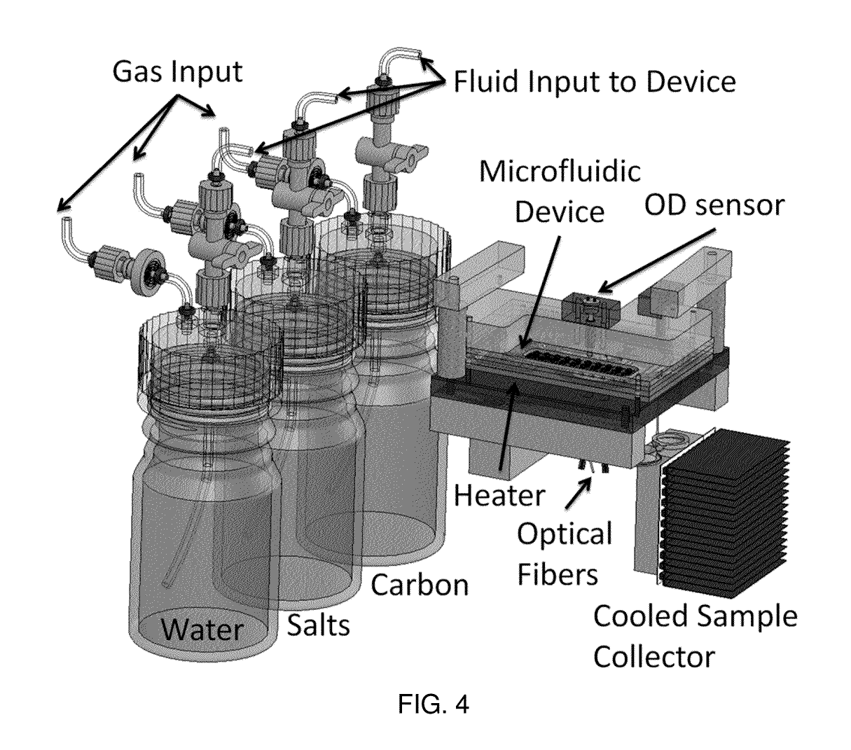

The supporting system for running the continuous culture included the external fluid supplies, pneumatic actuators, optical sensor electronics, temperature controller, and bioreactor control software for controlling oxygen, cell density, and flow rate. The device interface platform consisted of a circuit board heater in direct contact with the microfluidic chip and a mounting mechanism to maintain contact and align the chip with the required optical sensors as shown in FIG. 4. The chip output was connected to a thermal electrically cooled 1.5 mL Eppendorf tube for sample collection and chip inputs were connected to pressurized external fluid bottles.

Liquid supply reservoirs were fabricated out of standard GL45 capped glass jars by drilling holes and integrating threaded hose barbs for pressure input and fluid output. Fluids were pressurized through 0.22 μm filters (Pall Corporation) and extracted through Tygon tubing (S-50-HL) at the base of each jar. Fluidic interfaces were integrated directly onto the microfluidic chips by machining hose barbs at each fluid input and output.

Pneumatic actuation was performed using miniature 3-way solenoid switches (The Lee Co., LHDA0521111H) driven by digital driver circuits (Freescale MCZ33879). Interfaces for the pressurized gas inputs were also machined directly into each microfluidic device allowing reuse of a single 20 tube press fit connector.

Optical sensors were addressed by PMMA fiber bundles made from a central 1-mm excitation fiber (Industrial Fiber Optics, IF-C-UIOOO) and nine surrounding 500-μm collection fibers (IF-C-U500). Excitation fibers were split in the center and placed into a mount allowing integration of color glass filters (CYI Laser, BG3 and BG39). Optical density was measured through a transmission configuration incorporating low numerical aperture optics that permitted a linear correlation between optical density and cell density up to (1 cm OD_{600 nm}>50).

The optical collection fibers terminated at silicon photodetectors connected to transimpedance amplifiers. For oxygen sensors, long pass filters (CYI laser, RG9) were used, and for optical density and pH sensors, shorter wavelength long pass filters were used (KOPP 3482). Plastic mounts allowed integration and alignment of collection fibers, filters, so and photodetectors. Photodetectors, transimpedance amplifiers, and analog to digital converters were integrated onto circuit boards for direct digital readout.

Heaters and digital temperature sensors (LM9523I) were directly integrated on circuit boards and mounted at the base of each device. All digital control of solenoid drivers, data acquisition, and temperature were performed by a field programmable gate array (FPGA) (Opal Kelly XEM3100-1500P). System control was performed in MATLAB which measured and processed optical sensor data and ran control loops for oxygen and flow rate.

### Environmental Sensors

Oxygen Sensors:

Devices incorporated optical oxygen sensor spots in the base of the growth chamber sections as shown in FIG. 3B. Dissolved oxygen sensors were fabricated using platinum(II) octaethylporphine-ketone (PtOEPK) embedded in polystyrene and immobilized on glass disks. Sensor spots were calibrated by supplying different ratios of air and nitrogen with regulators and measuring phase. Extracted time constants were similar to other measurements (see D. B. Papkovsky et al., Analytical Chemistry, 1995, 67, 4112-4117) and have been reported previously (see K. S. Lee et al., Lab Chip, 2007, 7, 1539-1545). No sensor drift was observed for 3 weeks of continuous use and was tested by measuring minimum and maximum phase responses before and after growth experiments. Measurements of oxygen transfer rate were performed through the dynamic gassing method (see V. Linek et al., Chem. Eng. Technol., 1989, 12, 213-217) by step changing the mixing gas from helium to air and measuring the time constant for oxygen delivery into the reactor using the optical oxygen sensor.

pH Sensors:

pH sensor spots were purchased from Presens GmbH. While pH sensors were commercially precalibrated, additional calibration of pH sensor phase was performed by measuring sensor phase when exposed to buffer solutions varying from pH 5 to pH 10. Due to the potential for pH sensor drift through photobleaching, medium was off-line sampled daily and pH was referenced to a commercial measurement system (Microelectrodes Inc. MI-410) to ensure accurate on-line measurements.

OD Sensors:

OD sensors consisted of 590 nm LEDs directly illuminating through the microfluidic device and captured by 500-μm collection fibers located directly under the device. To maintain fixed volume for optical density measurements, density was measured in the rigid pass through channel and a rigid connecting growth chamber channel with pathlengths of 850 μm and 116 μm to accommodate different cell densities. For the smaller OD values used for the continuous culture experiment, data from the longer 850-μm pathlength was used and resulted in an OD resolution of ±0.013 and ±0.02 OD units at OD=1 and OD=2 respectively due to noise in the measurement electronics. Absolute OD was calibrated by measuring different cell concentrations from the microreactor and comparing to commercial spectrophotometer data (Spectronic 20 Genesys) from concurrent 100-μL output samples. Comparison of on-line measurements and off-line measurements resulted in an accuracy for on-line OD measurements of ±0.09 OD units for differences between online and off-line data averaged over 288 hours.

HPLC Analysis:

For HPLC measurements, 50 to 100 μL of the continuous culture output was collected, centrifuged, and frozen immediately. For steady state measurements, HPLC samples were taken after steady state was reached. For dynamic experiments, HPLC samples were taken every 10 minutes by the cooled sample collector, centrifuged, and frozen. HPLC analysis was performed off-line on all samples after finishing the continuous culture experiment.

### Control Algorithms

Temperature Control:

Temperature was controlled through a closed loop PID controller between the FPGA and the digital temperature sensor mounted at the base of the device. The refresh rate of the controller was 13 Hz and the step response of the closed loop controller was approximately 2 minutes.

Dissolved Oxygen Control:

Dissolved oxygen was controlled by varying the oxygen concentration of the growth chamber peristaltic mixer actuation gas. A solenoid valve upstream of the mixer control solenoid valves adjusted the input gas concentration by varying the duty cycle of two input gases, oxygen and helium at 3 psi. The valve actuation period was set to 10 Hz and alternated between either an oxygen or helium humidification reservoir. The duty cycle of the switch was controlled by a computer which periodically polled the FPGA for optical sensor data at a period of 30 seconds and ran a proportional-integral control algorithm based on the error between the measured oxygen and the oxygen setpoint.

Flow Control:

The flow rate was controlled by the computer system using optical density data measured at a 30 second period. For turbidostat control, a simple on-off control algorithm was used where the flow rate was set to either a high or low value depending on an optical density threshold. For chemostat control and feed control, the flow rate was set open loop by the software to a programmed injection rate versus time profile.

Escherichia coli Culture

Inoculation:

The full deflection mixer allowed automatic removal of most of the fluid volume in the growth chamber. To inoculate, One output valve was opened (B4=O) and all three growth chamber sections were pressurized to remove the internal air and reduce the chamber volume to nearly zero. Then a sterile tube was connected to the output to introduce inoculum. For inoculation, two of the growth chamber sections were depressurized, allowing them to back fill to 1 mL from the inoculation tube while still preventing the third growth chamber section from filling. The output port was then closed to seal the chamber (B4=P). If any air bubbles remained in the reactor, the inoculation procedure could be repeated indefinitely until all of the internal air bubbles were removed.

Cell and Medium Preparation:

Escherichia coli FB21591 (thiC::TnS-pKD46, Kan^{R}), obtained from the E. coli Genome Project at the University of Wisconsin (http://www.genome.wisc.edu), was used in continuous culture experiments. Inocula for experiments was prepared by streaking LB (Luria-Bertani) plates with 100 mg/L Kanamycin from a frozen stock followed by 5 mL test tube growths at 37° C. in LB with 100 mg/L Kanamycin. After reaching stationary phase, cells were transferred into 5 mL of defined medium and grown again to stationary phase. A 5-mL inoculum at OD_{600 nm}=0.01 was prepared from the defined medium test tube culture for direct injection into the continuous culture microreactor. Previous measurements of optical density for this cell line resulted in a conversion factor to dry cell weight (dew) of 0.33 g-dcw/L/OD (see H. L. T. Lee et al., Lab Chip, 2006, 6, 1229-1235).

The defined medium for test tube cultures consisted of (per liter): 13.5 g KH₂PO₄, 4.0 g (NH₄)₂HPO₄, 1.4 g MgSO₄.H₂O, 1.7 g citric acid, 0.3 g thiamine, 5 g glucose, 10 mL trace metal solution, and 100 mg Kanamycin which were all filter sterilized and stored in an autoclaved glass bottle. The trace metal solution was composed of (per liter 5 M HCl): 10.0 g FeSO₄.7H₂O, 2.0 g CaCl₂, 2.2 g ZnSO₄.7H₂O, 0.5 g MnSO₄.4H₂O, 1.0 g CuSO₄.5H₂O, 0.1 g (NH₄)₆Mo₇O₂₄.4H₂O, and 0.02 g Na₂B₄O₇.H₂O.

Defined medium for continuous cultures were split into individual components. The same defined medium specified for test tube cultures but without glucose was placed in one feed bottle. Two other feed bottles were used, one containing DI water and one containing 10 g/L glucose, both of which were steam sterilized in an autoclave.

Sterilization:

To ensure sterility, devices were placed in heat sealed bags and gamma irradiated at 16 kGy. Medium components were mixed and then filter sterilized into autoclaved bottles. Separation of feed bottles into pure chemical components ensured that chemotaxis and feed bottle contamination were prevented since no feed bottle contained enough components to support cell growth and culture media was prepared on-chip. Tests for upstream contamination and growth chamber contamination were performed at the end of the experiment by streaking the initial culture with the harvested microbioreactor fluid from before the peristaltic pump and within the growth chamber.

### Results and Discussion

Mixing:

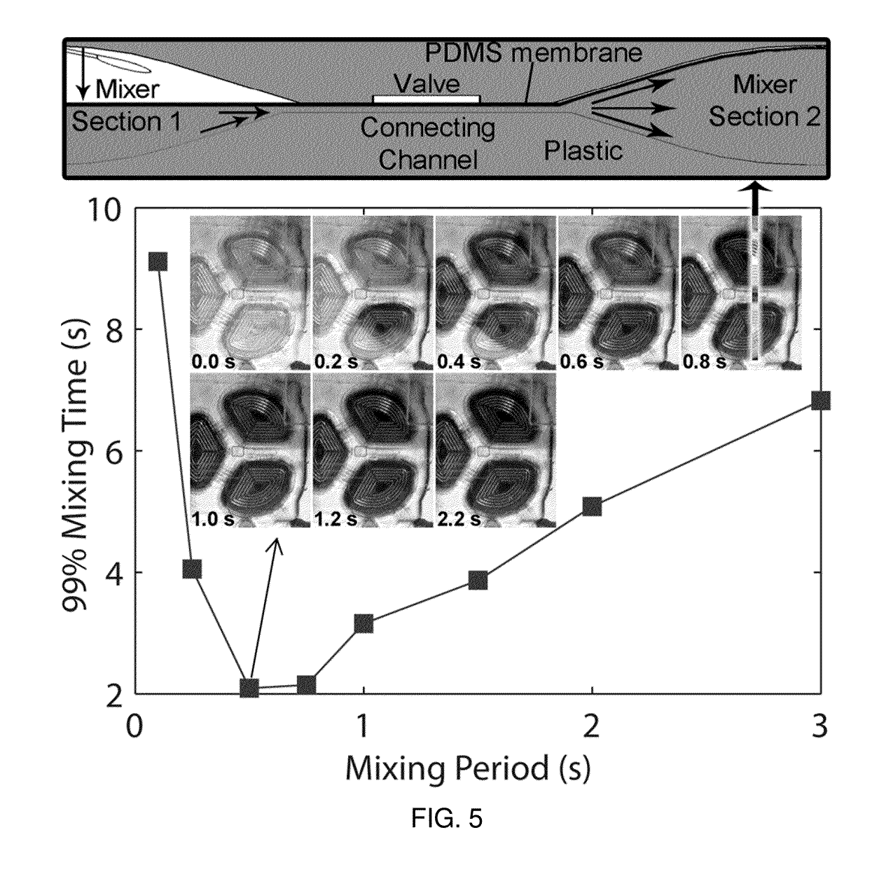

Mixing was characterized by measuring the contrast range of images taken with a digital camera (Opteon) for a solution of 0.3 mM bromothymol blue after addition of 0.1 M hydrochloric acid and sodium hydroxide by the peristaltic pump. Single exponential fits to the contrast change versus time resulted in a maximum mixing speed of 2 seconds at actuation conditions of 3 psi and 2 Hz full cycle. Mixing speed was highly dependent on actuation frequency, with a clear maximum efficiency at 2 Hz as shown in FIG. 5. Faster frequencies resulted in incomplete deflection and inefficient turbulent flow generation while slower frequencies resulted in full deflection faster than the actuation frequency and substantial wait time between states.

Fast homogenous mixing was possible by forcing fluid through small channels located between the mixer sections and by allowing full vertical deflection of the chamber from 0 mm to 2 mm forcing a large volume displacement with each stroke.

Oxygen Transfer Coefficient:

Unlike previously fabricated all-PDMS devices, oxygen transfer rates in many non-PDMS devices are generally not complicated by multiple paths for oxygen diffusion. In addition, the mixing times are generally fast enough to be approximate as instantaneous in comparison with the oxygen diffusion time. The differential equation governing oxygen diffusion into the reactor assumes perfect fluid mixing since the concentration of dissolved oxygen, C, is not a function of position.

$$\frac{\partial C}{\partial t} = {k}_{L} a ( {C}_{in} ( t ) - C ) - OUR \qquad [ 1 ]$$

Where k_{L}a is the oxygen transfer rate of the system, including diffusion through the PDMS, surface area of PDMS-water contact, and water volume; Cᵢₙ is the saturation concentration in the liquid for a given oxygen partial pressure, and OUR is the oxygen uptake rate. For a one-dimensional diffusion system with instantaneous mixing, this differential equation accurately describes the dynamics of oxygen in the liquid. Therefore, established methods for measuring k_{L}a such as the dynamic gassing method can be used to characterize the reactor.

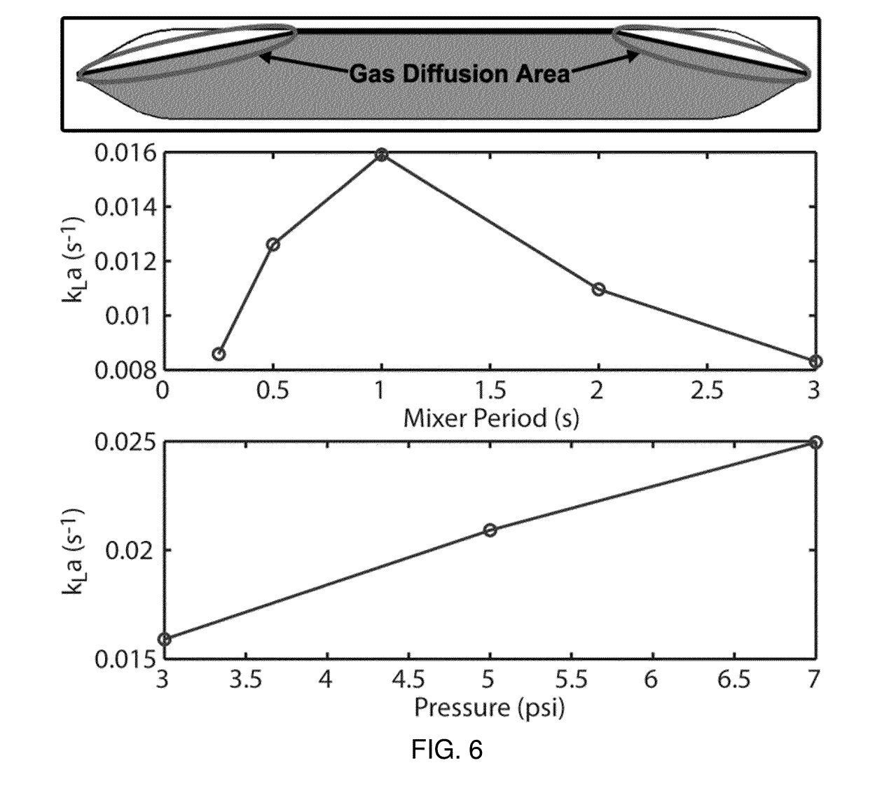

Dynamic gassing measurements in FIG. 6 show a maximum k_{L}a of 0.016 s⁻¹ and 0.025 s⁻¹ for input pressures of 3 psi and 7 psi, respectively. Since the membranes were capable of laminating both the upper and lower surface of the growth chamber, resulting in a decrease in contact area between the PDMS and the gas headspace, pressure dependence of k_{L}a was expected.

Since k_{L}a determines the maximum supported cell density in the reactor, it can be used to calculate the maximum OD supported by the system. Setting Equation 1 to steady state and assuming that the concentration of dissolved oxygen in the water is zero, we calculate the maximum cell density supported to be OD=14.7, assuming Cᵢₙ=1.26 mM for a maximum water solubility of pure oxygen at 37° C. and 3 psi, a maximum OUR=15.4 mmol O₂/g-dcw/h, 0.33 g-dcw/L/OD, and a k_{L}a=0.016 s⁻¹. Previously reported continuous culture systems did not require growth beyond OD=7.5 suggesting that the system is adequate for continuous culture experiments.

Flow Rate:

Flow rate through the peristaltic pump was characterized by attaching a capillary tube to the output of the device. A measurement system utilizing a triggered CCD camera (Opteon) and a 600-μm inner diameter glass capillary tube (McMaster 8729K57) was used, resulting in volume resolution of 18 nL per pixel. An image was captured every pump period and processed in MATLAB to determine the position of the meniscus.

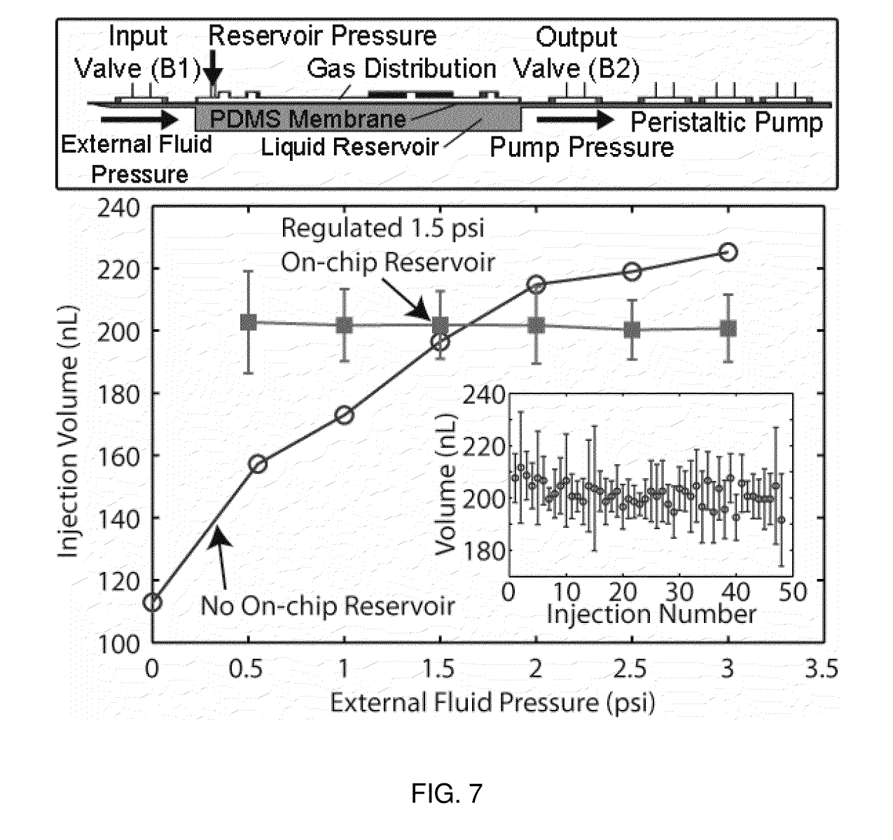

Flow rate through the peristaltic pump was characterized for various backpressures from the external fluid input. As shown in FIG. 7, the volume varies by nearly a factor of two for input pressure variations from 0 to 3 psi. Enabling the on-chip reservoir with a 1.5 psi pressure at the reservoir pressure input effectively eliminates these variations and maintains a consistent injection volume of 200 nL over the 3 psi range in external fluid pressure.

E. coli Continuous Culture:

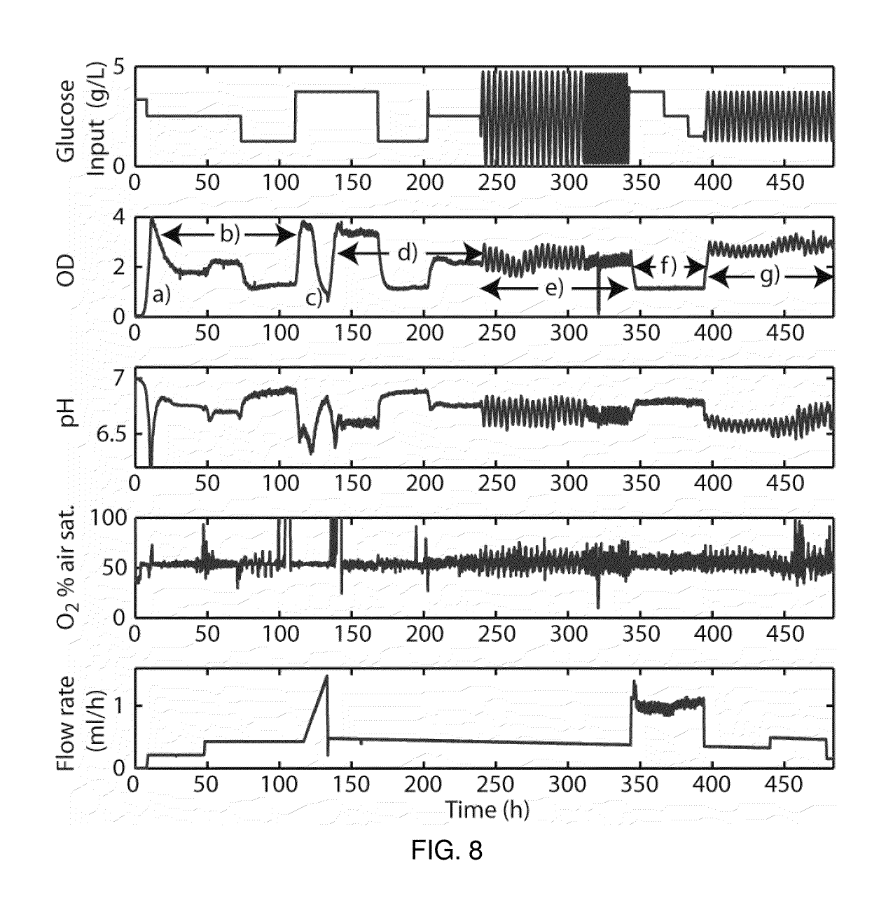

A 3-week long continuous culture experiment was performed using the bioreactor to demonstrate device operation and novel control conditions possible with the device which enable direct observation of cell metabolic processes. Since glucose and salts were separated into individual feed bottles, the peristaltic pump could vary the concentration of glucose in the feed. The glucose concentration was adjusted by changing the ratio of DI water to glucose injections while keeping the total injections equal to the salt injections. This prevented dilution of the salt media. Due to the ability to switch between multiple inputs and accurately measure optical density, pH, and oxygen, a variety of new functions were possible. Multiple experiments in chemostat and turbidostat modes with different media compositions could be run in a single device, modulation of input sources were possible, HPLC sample collection times were fast enough to look at dynamics, control of oxygen during continuous culture could now be implemented, and operation for 3 weeks without evaporation was possible, all while maintaining sterility. On-line growth data from the continuous culture is given in FIG. 8.

Initially, the cells were grown in batch to assess viability and oxygen transfer as shown in portion (a) of the plots in FIG. 8. This resulted in a significant decrease in pH typical of batch growths. Even at OD 4, the oxygen supply was sufficient to maintain an oxygen concentration of 50% air saturation. Then continuous culture was turned on to observe known E. coli metabolic functions. Chemostat operation was initiated at flow rates specified in the flow rate plot and the corresponding cell densities are given in portion (b) of the plots in FIG. 8. Consistent with previous continuous culture experiments, increases in the flow rate at 50 hours resulted in higher optical density at the same glucose concentration.

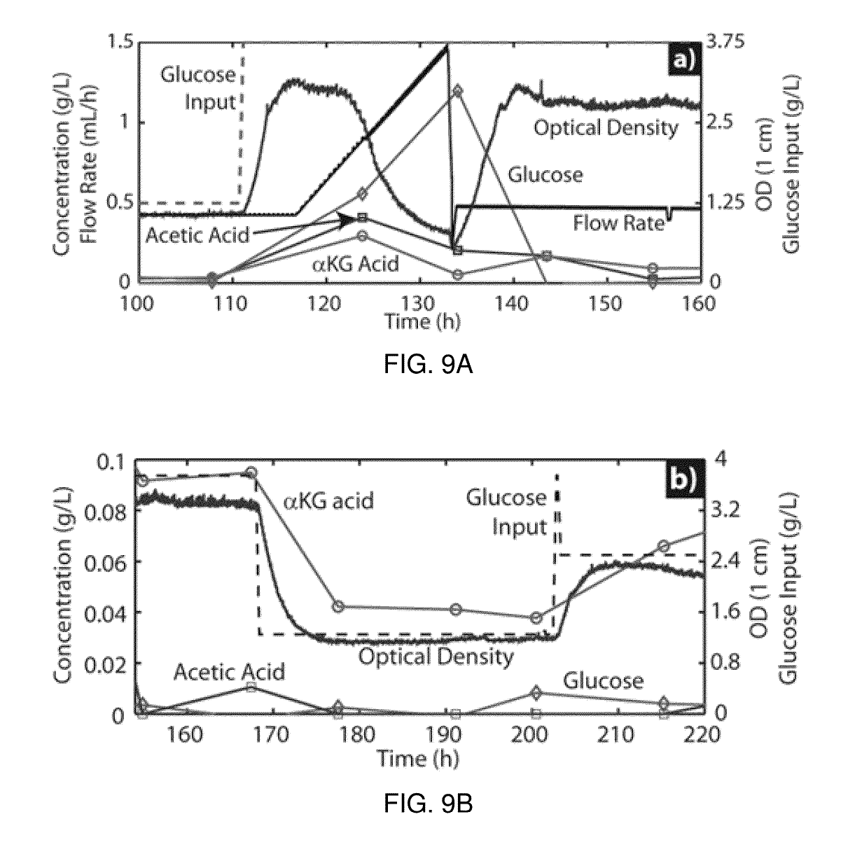

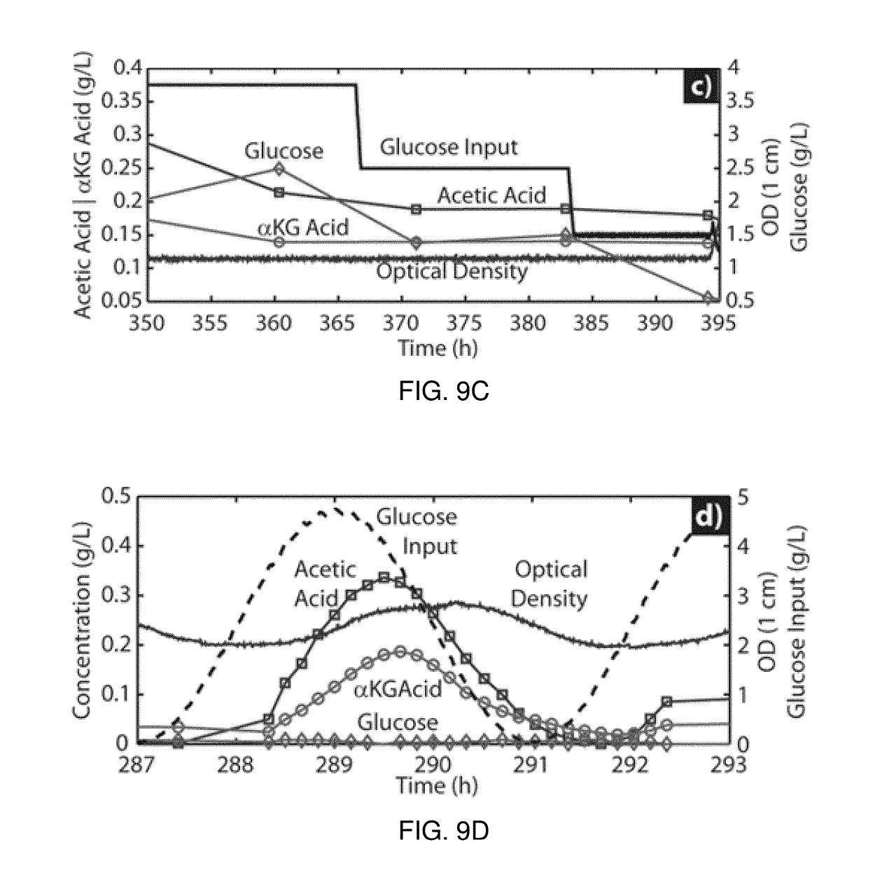

At 120 hours in portion (c) of the plots in FIG. 8, the flow rate was ramped up to induce washout. This allows us to sweep the flow rate to approximately find the maximum growth rate. If one estimates the maximum growth rate as the point at which the cell density starts to decrease, a maximum growth rate of 0.85 h⁻¹ was determined. From HPLC sampling during washout given in FIG. 9A, acetic acid and glucose accumulation was observed when the cell density started to decrease, typical of overflow metabolism. Restoration of chemostat operation at 155 hours resulted in complete removal of glucose and acetate from the medium.

After washout, three steady states at different input glucose concentrations demonstrated that the cell density could be controlled by changing the glucose concentration, as shown in portion (d) of the plots in FIG. 8. The cells grow in direct proportion to the glucose input as expected for chemostat operation, with optical densities of 1.16±0.024, 2.22±0.074, and 3.33±0.040 for input glucose concentrations of 1.25 g/L, 2.5 g/L. and 3.75 g/L, respectively. Referring to the HPLC data in FIG. 9B, one can see that in chemostat operation, the acid production was very different from what one would observe in washout conditions. Instead of the cells producing acetic acid, the majority of acid production was alpha-ketoglutaric acid and succinic acid. Since the quantities of each track almost identically throughout the growth, only aKG acid is shown. The production of aKG acid was also proportional to the cell density, with an average production rate of 0.042±0.005 g/g-dcw/h suggesting that the production rate per cell was not actually changing with glucose input. Chemostat experiments using feed control demonstrate the ability to control the cell density on-line by varying the glucose concentration.

In contrast to chemostat operation, turbidostat operation allows one to study the metabolic behavior of cells in washout conditions such as overflow metabolism and maximum growth rate in steady state. Turbidostat operation is shown in portion (f) of the plots in FIG. 8. Cells were maintained at an OD of 1.14±0.013, demonstrating closed loop control of OD to within 1.2%. Looking at the flow control variable, one can extract a maximum growth rate of rate of 0.994±0.051 h⁻¹. This value was higher than estimations from washout, demonstrating that washout underestimates the maximum cell growth rate. In addition to flow control, since one can change the glucose input concentration without affecting the flow rate, one can observe overflow metabolism directly. From the HPLC data shown in FIG. 9C, during turbidostat operation, one can see that acetate production increases as the glucose concentration so in the reactor increases. Alpha-ketoglutaric acid production, which was proportional to cell density in chemostat operation, also did not change in turbidostat operation at constant OD. However, the amount of acid produced was higher at 0.368±0.026 g/g-dcw/h. This could reflect an increase in cell metabolism and the citric acid cycle during turbidostat operation.

While steady state operation can allow one to probe cell metabolism through mass balances, dynamic operation can also enable probing of how cells respond dynamically to changes in input concentrations. As shown in portions (e) and (g) of the plots shown in FIG. 8, individual component control at the input allowed for programmed input dynamics such as sinusoidal modulation of glucose at different frequencies. This type of operation could be used to study time responses of different metabolic pathways. Since the reactor volume was 1 mL, high speed sampling for HPLC analysis could also be performed, resulting in high resolution data of the chemical responses to input feed modulation as shown in FIG. 9D.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1-54. (canceled)

55. An article for transporting liquid, comprising:
a first chamber comprising a first wall and a second wall, with at least a portion of the first wall of the first chamber defined by a first flexible member portion;
a first inlet and a first outlet fluidically connected to the first chamber;
a second chamber comprising a first wall and a second wall, with at least a portion of the first wall of the second chamber defined by the first flexible member portion;
a second inlet fluidically connected to the second chamber;
a third chamber comprising a first wall and a second wall, with at least a portion of the first wall of the third chamber defined by a second flexible member portion;
a third inlet and a third outlet fluidically connected to the third chamber;
a fourth chamber comprising a first wall and a second wall, with at least a portion of the first wall of the fourth chamber defined by the second flexible member portion;
a fourth inlet fluidically connected to the fourth chamber;
a first fluidic pathway connected to the first outlet and the third inlet;
a first valve fluidically connected to the first chamber and to the first inlet;
a second valve fluidically connected to the third chamber and to the third outlet;
a bypass channel fluidically connected to the first valve and the second valve;
a third valve fluidically connected to the bypass channel and a fluid source; and
a fourth valve fluidically connected to the bypass channel and an article outlet;
wherein:
when a pressure within the first chamber is greater than a pressure within the second chamber by a first threshold pressure difference, the first flexible member portion contacts the second wall of the second chamber;
when a pressure within the third chamber is greater than a pressure within the fourth chamber by a second threshold pressure difference, the second flexible member portion contacts the second wall of the fourth chamber;
when the first and second valves are closed, the first and third chambers are fluidically isolated from the bypass channel and the fluid source;
when the third valve is closed, the first and third chambers are fluidically isolated from the fluid source; and
when the first and second valves are opened, the bypass channel forms at least a portion of a second fluidic pathway fluidically connecting the first chamber and the third chamber.

56. An article for transporting liquid, comprising:
an article fluid inlet;
an article fluid outlet;
a first chamber comprising a first wall and a second concave wall, with at least a portion of the first wall of the first chamber defined by a first flexible gas permeable membrane portion;
a first inlet and a first outlet fluidically connected to the first chamber;
a second chamber comprising a first wall and a second concave wall, with at least a portion of the first wall of the second chamber defined by the first flexible gas permeable membrane portion;
a second inlet fluidically connected to the second chamber;
a third chamber comprising a first wall and a second concave wall, with at least a portion of the first wall of the third chamber defined by a second flexible gas permeable membrane portion;
a third inlet and a third outlet fluidically connected to the third chamber;
a fourth chamber comprising a first wall and a second concave wall, with at least a portion of the first wall of the fourth chamber defined by the second flexible gas permeable membrane portion;
a fourth inlet fluidically connected to the fourth chamber;
an interconnect channel connected to the first outlet and the third inlet;
a first fluidic pathway fluidically connected to the first outlet and the third inlet;
a first valve fluidically connected to the first chamber and to the first inlet;
a second valve fluidically connected to the third chamber and to the third outlet; and
a bypass channel fluidically connected to the article inlet, the article outlet, the first inlet, and the third outlet;
wherein:
when the first and second valves are closed, the first and third chambers are fluidically isolated from the bypass channel;
when the first and second valves are opened, the bypass channel forms at least a portion of a second fluidic pathway fluidically connecting the first chamber and the third chamber;
the average depth of the first chamber is at least 400% larger than the minimum cross sectional dimension of at least one of the first inlet and the first outlet; and
the average depth of the third chamber is at least 400% larger than the minimum cross sectional dimension of at least one of the third inlet and the third outlet.

57. A method, comprising:
providing a device comprising:
a first chamber comprising a first wall and a second wall, with at least a portion of the first wall of the first chamber defined by a first flexible member portion;
a first inlet and a first outlet fluidically connected to the first chamber;
a second chamber comprising a first wall and a second wall, with at least a portion of the first wall of the second chamber defined by the first flexible member portion;
a second inlet fluidically connected to the second chamber;
a third chamber comprising a first wall and a second wall, with at least a portion of the first wall of the third chamber defined by a second flexible member portion;
a third inlet and a third outlet fluidically connected to the third chamber;
a fourth chamber comprising a first wall and a second wall, with at least a portion of the first wall of the fourth chamber defined by the second flexible member portion;
a fourth inlet fluidically connected to the fourth chamber;
a first fluidic pathway connected to the first outlet and the third inlet;
a first valve fluidically connected to the first chamber and to the first inlet;
a second valve fluidically connected to the third chamber and to the third outlet;
a bypass channel fluidically connected to the first valve and the second valve;
a third valve fluidically connected to the bypass channel and a fluid source; and
a fourth valve fluidically connected to the bypass channel and an article outlet;
wherein:
when a pressure within the first chamber is greater than a pressure within the second chamber by a first threshold pressure difference, the first flexible member portion contacts the second wall of the second chamber;
when a pressure within the third chamber is greater than a pressure within the fourth chamber by a second threshold pressure difference, the second flexible member portion contacts the second wall of the fourth chamber;
when the first and second valves are closed, the first and third chambers are fluidically isolated from the bypass channel and the fluid source;
when the third valve is closed, the first and third chambers are fluidically isolated from the fluid source; and
when the first and second valves are opened, the bypass channel forms at least a portion of a second fluidic pathway fluidically connecting the first chamber and the third chamber;
applying a first gas pressure to the second chamber;
applying a second gas pressure to the second chamber;
applying a third gas pressure to the fourth chamber;
applying a fourth gas pressure to the fourth chamber
closing the first and second valves;
opening the third and fourth valves;
introducing a fluid into the bypass channel;
closing the third and fourth valves after introducing the fluid into the bypass channel;
opening the first and second valves after closing the third and fourth valves; and
wherein the first gas pressure is greater than the second gas pressure.

58. The article of claim 55, wherein when the difference between a pressure within the first chamber and a pressure within the second chamber is at least one value below 5 psi, the first flexible member portion essentially consistently contacts the second wall of the second chamber.

59. The article of claim 55, wherein when the difference between a pressure within the first chamber and a pressure within the second chamber is at least one value below 3 psi, the first flexible member portion essentially consistently contacts the second wall of the second chamber.

60. The article of claim 55, wherein when the difference between a pressure within the first chamber and a pressure within the second chamber is at least one value below 1 psi, the first flexible member portion essentially consistently contacts the second wall of the second chamber.

61. The article of claim 55, wherein the second inlet is configured to deliver a gas to the second chamber.

62. The article of claim 55, wherein the first and second flexible member portions comprise a gas-permeable medium.

63. The article of claim 62, wherein the gas-permeable medium comprises a gas-permeable polymer.

64. The article of claim 63, wherein the gas-permeable polymer comprises a silicon-based polymer.

65. The article of claim 64, wherein the silicon-based polymer comprises polydimethylsiloxane.

66. The article of claim 62, wherein the flexible member is configured to transport a gas into the chamber.

67. The article of claim 66, wherein the gas is used as a reactant in a chemical and/or biochemical reaction within the chamber.

68. The article of claim 55, wherein the article is configured to perform a chemical and/or biochemical reaction.

69. The article of claim 68, wherein the article is configured to perform cell culture.

70. The article of claim 55, wherein the bypass channel has substantially rigid walls.

71. The article of claim 55, wherein the article comprises at least one microfluidic channel.

72. The article of claim 55, wherein at least one of the first chamber and the third chamber comprises cell culture medium.

73. The article of claim 55, wherein at least one of the first chamber and the third chamber comprises a biological cell.

74. The article of claim 55, wherein:
the average depth of the first chamber is at least 400% larger than the minimum cross sectional dimension of at least one of the first inlet and the first outlet; and
the average depth of the third chamber is at least 400% larger than the minimum cross sectional dimension of at least one of the third inlet and the third outlet.
